(19) United States
(12) Patent Application Publication
(10) Pub. No.: US 2008/0300661 A1
(43) Pub. Date: Dec. 4, 2008

(54) Perceptible Apparatus and Methods for Reactive Effect

(75) Inventors: Bertrand Babinet, Lyons, CO (US); David A. Thomas, Longmont, CO (US); Jon Tempest, Henderson, NV (US)

Correspondence Address: JACKSON ESQUIRE;ROGER A. JACKSON, 1115 GRANT STREET, SUITE G-7, DENVER, CO, 80203-2399, US

(73) Assignee: STAR ENERGETICS HOLDING COMPANY, Longmonth, CO (US)

(21) Appl. No.: 10/596,376

(22) PCT Filed: Dec. 10, 2004
(86) PCT No.: PCT/US2004/041238

§ 371 Date: May 12, 2007

Related U.S. Application Data
(60) Provisional application No. 60/529,462, filed on Dec. 11, 2003.

(51) Int. Cl. A61N 5/06

(52) U.S. Cl. 607/88

(57) ABSTRACT

A perceptible apparatus and methods for use and calibration in helping to create a reactive effect upon a user is disclosed. The present invention includes control circuitry operative to generate a one of a plurality of selected signals. Also included is structure for producing a variable perceptible output in response to one of a plurality of the selected signals and a translucent element adjacent to the structure for producing a variable perceptible output, the translucent element is operative to diffuse and emit the variable perceptible output.

### RELATED PATENT APPLICATIONS

This patent application claims priority from a Patent Cooperation Treaty patent application having international application number PCT/US04/41238 filed on Dec. 10, 2004 by Star Energetics Holding Company, L.L.C. that claims priority from a U.S. provisional patent application Ser. No. 60/529,462 filed on Dec. 11, 2003 by David Thomas of Longmont, Colo., Bertrand Babinet of Lyons, Colo., and Jon Tempest of Parker, Colo.

### TECHNICAL FIELD

The present invention relates to a device that creates a subtle-energy field, similar in nature to the bio energetic field of the human body. This apparatus and associated methods have a number of applications, including holistic health/energy-balancing modalities.

### BACKGROUND OF INVENTION

This invention is applicable for many types of energetic treatment modalities. Although subtle-energy (the basis of these modalities) is not yet measurable by conventional, scientific means, its existence is widely accepted among holistic-health practitioners. Homeopathic remedies are an example of a widely used modality, which is based on subtle energy. Many practitioners and other practitioner 55 or user 54 claim varying degrees of ability to “sense” subtle energy.

In addition to subtle-energy modalities, there are other modalities that use colored lights. Often, the lights are colored with the seven colors of the rainbow, being red, orange, yellow, green, blue, indigo, and violet. In many holistic modalities, it is believed that these seven colors of the rainbow map to certain energy centers of the body, called chakras.

There are a number of devices on the market that are used in the holistic-health field. Some of them generate colored light; some of them claim to generate a subtle energy field; some work in other ways. Many of them are quite expensive, in the range of $1,000 to as much as $18,000. Typically, the more expensive devices are owned by practitioners. User 54s must schedule an appointment with their practitioner in order to use a device. Additionally, the devices on the market frequently support only one treatment modality. This fact prevents them from becoming widely used, and keeps them expensive, as not many units are made and sold. What is needed is a low-cost, flexible, effective device that practitioners can sell, rent, or otherwise provide to their user 54s for home-use. This invention presents a breakthrough in energetic treatment in that it is a low-cost technology, highly effective, user-friendly, and programmable/customizable to support a variety of treatment modalities.

Users of the exemplary embodiment of the invention have experienced relief from a variety of physical discomforts, including headaches, back aches, neck/knee/shoulder pain, carpal tunnel syndrome, nausea, fatigue, and PMS. Early experiments suggest that the invention may also have utility in resolving psychological issues, as well.

U.S. Pat. No. 5,947,908 to Morris discloses an apparatus for deriving colors that have either a positive or negative impact upon an individual. Morris uses biofeedback means to determine if a color presented to a particular individual is healthful or stress producing. Morris uses color but does not have any subtle energy producing means. U.S. Pat. No. 4,940,323 to Downing discloses a visible light based stimulator device. Downing has a brief discussion about a technology for generating photons that “have a great affinity to affect the human body by interaction with the body's own biomagnetic field”. Downing's technology is very different from that used in the present invention, however, it does present another example of a patent which references the notion of subtle energy. U.S. Pat. No. 6,016,450 to Crock presents a treatment method and apparatus that is vastly different from the present invention, but includes an excellent history of research into the human aura (biofield). U.S. Pat. No. 6,602,275 to Sullivan discloses a device that uses a plurality of flashing LEDs to produce a healing effect on an individual, but does not have a means of transmuting the light energy generated into a subtle energy field. Sullivan requires a great many LEDs to be effective. Compared to the aforementioned prior art, the present invention has the advantages of low cost, portability and substantial effectiveness.

### SUMMARY OF INVENTION

Broadly, the present invention is a perceptible apparatus for use in helping to create a reactive effect upon a user, the present invention includes control circuitry operative to generate a one of a plurality of selected signals that are preferably pulse width modulation signals or alternative signals. Also included is a means for producing a variable perceptible output in response to one of a plurality of selected pulse width modulation signals and a translucent element adjacent to the means for producing a variable perceptible output, the translucent element is operative to diffuse and emit the variable perceptible output. Note that “perceptible output” includes both light and subtle energy as described herein.

In addition a method of using a perceptible apparatus for use in helping to create a reactive effect upon a user, comprises the steps of, positioning the user, providing the perceptible apparatus that includes, control circuitry operative to generate a one of a plurality of selected signals. Also provided is a means for producing a variable perceptible output in response to one of a plurality of selected signals, and a translucent element adjacent to the means for producing a perceptible output, with the translucent element being operative to diffuse and emit the perceptible output. A further step is in locating the perceptible apparatus to a selected position adjacent to the user, continuing to a step of activating the perceptible apparatus that is operational to illuminate the perceptible output in response to one of a plurality of selected signals. Also a step is in determining the characteristics of the selected signals to produce the desired effect.

Further, a method for calibrating a perceptible apparatus for use in helping to create a reactive effect upon a user, comprising the steps of, providing the perceptible apparatus that includes, control circuitry operative to generate singularly or simultaneously in each one of a plurality of selected modes a plurality of selected signals for each the mode, a plurality of LEDs, with each one LED variably illuminated in response to the plurality of selected signals for each one of a plurality of selected modes, and a translucent element adjacent to the plurality of LEDs, the translucent element is operative to diffuse and emit one light illuminated or a plurality of lights illuminated, all being operative to generate a subtle energy field, and further including calibration circuitry for each one of the LED's that is operational to help make brightness of each one of the LED's consistent amongst the plurality of different LEDs. Another step of providing a calibration device that includes a shroud that occludes substantially all external environment light from the translucent element and calibration device circuitry that provides a signal that is displayed indicating brightness and/or color of the LEDs. A further step of adjusting brightness using the calibration circuitry and calibration device display that is operational to further help make brightness of each one of the LED's consistent amongst the plurality of different LEDs from one unit to the next. A yet further step of adjusting color by using the calibration device display by replacing a selected LED that is operational to further help make color of each one of the LED's consistent amongst the plurality of different LEDs from one unit to the next.

These and other objects of the present invention will become more readily appreciated and understood from a consideration of the following detailed description of the exemplary embodiment(s) of the present invention when taken together with the accompanying drawings, in which;

### BRIEF DESCRIPTION OF DRAWINGS

FIG. 1 is a perspective view of the exemplary embodiment of the present invention;

FIG. 2 is a perspective view of the exemplary embodiment of this invention, with the translucent element 31 removed, to show how it plugs into the translucent element slot 47 in the top of housing 33, the power plug 35 is removed, exposing the power jack 48;

FIG. 3 is a perspective view of the exemplary embodiment of the present invention with the housing 33 removed, some of the parts on the circuit board 43 are omitted for clarity, translucent element 31 is elevated in this drawing, exposing the slots 49 in the translucent element supports 41 which properly locate the translucent element 31 with respect to the LEDs 44, 45, and 46;

FIG. 4 is a side view of the exemplary embodiment of the present invention with housing 33 removed, some of the parts on the circuit board 43 are omitted for clarity, translucent element 31 is shown resting in the slots 49 in translucent element supports 41, which properly locate the translucent element 31 with respect to the LEDs 44, 45, and 46;

FIG. 5 is a side view of the translucent element support 41 showing the translucent element alignment slot 49 and circuit board mounting slot 52;

FIG. 6 is a rear view of the exemplary embodiment of the present invention with housing 33 removed, some parts of the circuit board 43 are omitted for clarity, the translucent element 31 is shown resting in the slots 49 in the translucent element supports 41, in its proper relationship to LEDs 44, 45, and 46;

FIG. 7 is a schematic diagram of the circuit used in the exemplary embodiment of the present invention;

FIG. 8 is a perspective view of the exemplary embodiment of the present invention and the calibration device 121, the calibration device 121 is in position to be inserted into the exemplary embodiment of the present invention for the purpose of calibration;

FIG. 9 is an exploded perspective view showing the major components of the exemplary embodiment of the calibration device 121;

FIG. 10 is a schematic diagram of the circuit used in the exemplary embodiment of the calibration device 121;

FIG. 11 is a flowchart depicting the main functional loop of the firmware in the exemplary embodiment of the present invention of the calibration device;

FIG. 12 is a flowchart depicting the algorithm used by the firmware in the exemplary embodiment of the calibration device to determine the brightness of the red, green, and blue channels, and thus the color and brightness of light being measured;

FIG. 13 is a flow chart depicting the main functional loop of the firmware in the exemplary embodiment of the present invention;

FIG. 14 is a flowchart depicting the algorithm used by the firmware in the exemplary embodiment of the present invention to determine duty cycles of red, green, and blue based upon selected settings;

FIG. 15 is a flowchart depicting the process the present invention uses to determine duty cycles red, green, and blue based upon the knob settings;

FIG. 16 is a flowchart depicting the algorithm or firmware of the exemplary embodiment of the present invention uses to determine the proper mode being clearing or manual;

FIG. 17 is a flowchart depicting the algorithm the exemplary embodiment of the present invention uses to calculate the duty cycles for clearing mode;

FIG. 18 is a block diagram depicting the major components of the exemplary embodiment of the present invention;

FIG. 19 is an example of a symmetrical pattern of operation used for the clearing mode in the exemplary embodiment of the invention;

FIG. 20 depicts a user 54 using an exemplary embodiment of the present invention;

FIG. 21 depicts a user 54 using an exemplary embodiment of the present invention in front of a reflective mirror 66;

FIG. 22 is a flowchart depicting the steps for using an exemplary embodiment of the present invention in manual mode;

FIG. 23 is a flowchart depicting the steps for using an exemplary embodiment of the present invention in clearing mode;

FIG. 24 is a flowchart depicting the steps for using an exemplary embodiment of the present invention in a program mode;

FIG. 25 is a flowchart depicting the steps for using the present invention to replicate a particular energetic effect;

FIG. 26 is a perspective view of the exemplary embodiment of the present invention with a container of liquid 80 in position to be encoded with a subtle energy;

FIG. 27 is a perspective view of the exemplary embodiment of the present invention with a crystal 81 in position to be energetically cleared;

FIG. 28 shows the measurements of the translucent element 31 that are used in the exemplary embodiment of the program mode example; and

FIG. 29 is a flowchart showing the calculation of duty cycles for the chakra modes.

### REFERENCE NUMBER IN DRAWINGS

30. Perceptible apparatus

31. Translucent element

32. Display

33. Common housing

34. Wall Transformer

35. Power Plug

36. Knob 1 (red)

37. Knob 2 (green)

38. Knob 3 (blue)

39. Knob 4 (mode-select)

40. Housing bottom

41. Translucent element support

42. Display standoffs

43. Circuit board

44. Red LED

45. Green LED

46. Blue LED

47. Translucent element slot

48. Power jack

49. Translucent element alignment slot

51. Circuit

52. Circuit board mounting slot

54. User

55. Practitioner

60. Control circuitry

62. Pulse width modulation signals

64. Means for producing a variable perceptible output

65. Diffused and emitted variable perceptible output

66. Reflective mirror

68. Calibration circuitry

70. Mode select circuitry

71. LED to translucent element clearance

72. Distance between the circuit board 43 and the lower edge of the translucent element 31

80. Liquid container

81. Crystal

### Reference Numerals Used in Calibration Device

121. Calibration device

122. Power jack—calibration device

123. Display—calibration device

124. Housing—calibration device

125. Calibration device circuitry

126. Translucent element shroud—calibration device

127. Ribbon cable—calibration device

128. Color sensor chip

129. Color sensor chip slot

130. Shroud half with cutaway

131. Shroud half without cutaway

132. Cutaway for translucent element

133. Ribbon cable slot

134. External environment light

### Reference Designators Used in Schematic Diagram of Perceptible Apparatus

U1 Microcontroller—Microchip Technologies—PIC 18F252-I/SP

U2 LCD Display—Lumex—LCM-S01602DTR/A

U3 Clock oscillator, ½ size, 20 MHZ—Fox—H5C2E-200

U4 Quad op-amp—Microchip Technologies—MCP604-I/P-ND

U5 Voltage Regulator, 5V—On semi—MC78L05ACP

C1 0.1 uf capacitor

C2 0.1 uf capacitor

C3 100 uf electrolytic capacitor

C4 0.1 uf capacitor

Q1 Power mosfet transistor, logic level—Fairchild Semiconductor—NDP4060L

Q2 Power mosfet transistor, logic level—Fairchild Semiconductor—NDP4060L

Q3 Power mosfet transistor, logic level—Fairchild Semiconductor—NDP4060L

D1 1 amp Diode

LED1 Blue Superflux LED—Lumileds—HPWN-MB00

LED2 Green Superflux LED—Lumileds—HPWN-MG00

LED3 Red Superflux LED—Lumileds—HPWT-RD00-0000

R1 10K ¼ watt

R2 5K pot—Xicon—(Mouser part number 317-2091-5K)

R3 5K pot—Xicon—(Mouser part number 317-2091-5K)

R4 5K pot—Xicon—(Mouser part number 317-2091-5K)

R5 5K pot—Xicon—(Mouser part number 317-2091-5K)

R6 22 ohm ½ watt

R7 0 ohm resistor

R8 22 ohm ½ watt

R9 0 ohm resistor

R10 3.3K ¼ watt

R11 3.3K ¼ watt

R12 10 ohm ½ watt

R13 22 ohm ½ watt

R14 10 ohm ½ watt

R15 1K trimpot, 20 turn—Vishay—(Mouser part number 72-T93XB-1K)

R16 1K trimpot, 20 turn—Vishay—(Mouser part number 72-T93XB-1K)

R17 0 ohm resistor

R18 3.3K ¼ watt

R19 1K trimpot, 20 turn—Vishay—(Mouser part number 72-T93XB-1K)

R20 10 ohm ½ watt

R21 20K trimpot (Mouser part number 531-PT10H-20K)

R22 4.7K ¼ watt

R23 4.7K ¼ watt

R24 4.7K ¼ watt

### Reference Designators Used in Schematic Diagram of Calibration Device

U10 Microcontroller—Microchip Technologies—PIC 18F252-I/SP

U20 LCD Display—Lumex—LCM-S01602DTR/A

U30 Clock oscillator, ½ size, 20 MHZ—Fox—H5C2E-200

U40 Color sensor chip—TAOS inc.—TCS230

U50 Voltage Regulator, 5V—On semi—MC78L05ACP

C10 0.1 uf capacitor

C20 0.1 uf capacitor

C30 100 uf electrolytic capacitor

C40 0.1 uf capacitor

D10 1 amp Diode

R100 10K ¼ watt

R200 20K trimpot (Mouser part number 531-PT10H-20K)

### DETAILED DESCRIPTION

Before disclosing the exemplary embodiment of the perceptible apparatus 30 in detail, it is to be understood that the perceptible apparatus 30 is not limited in its application to the details of the exemplary embodiment disclosed, since the perceptible apparatus 30 is capable of other embodiments. Also, the terminology used herein as for the purpose of description and not of limitation.

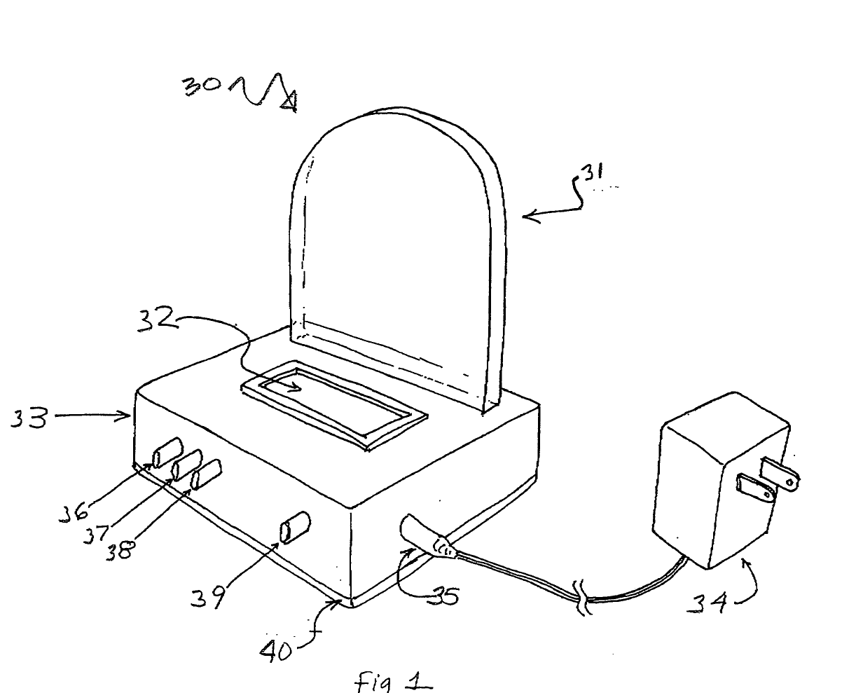

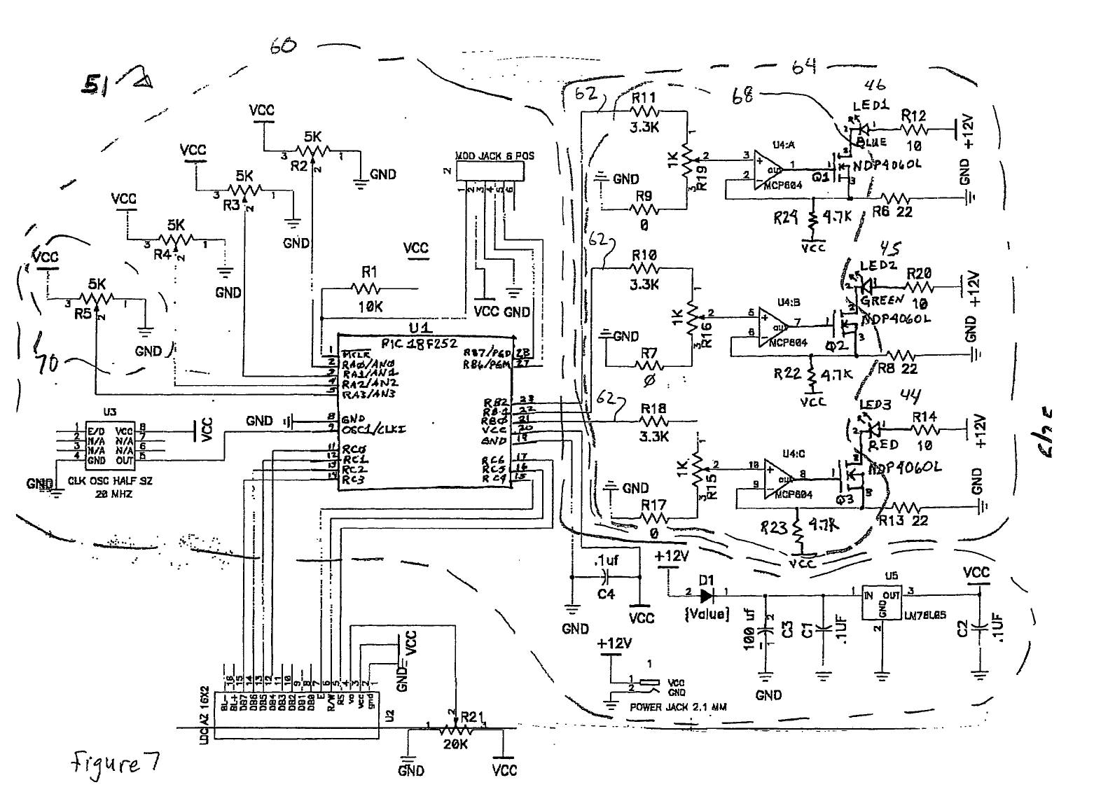

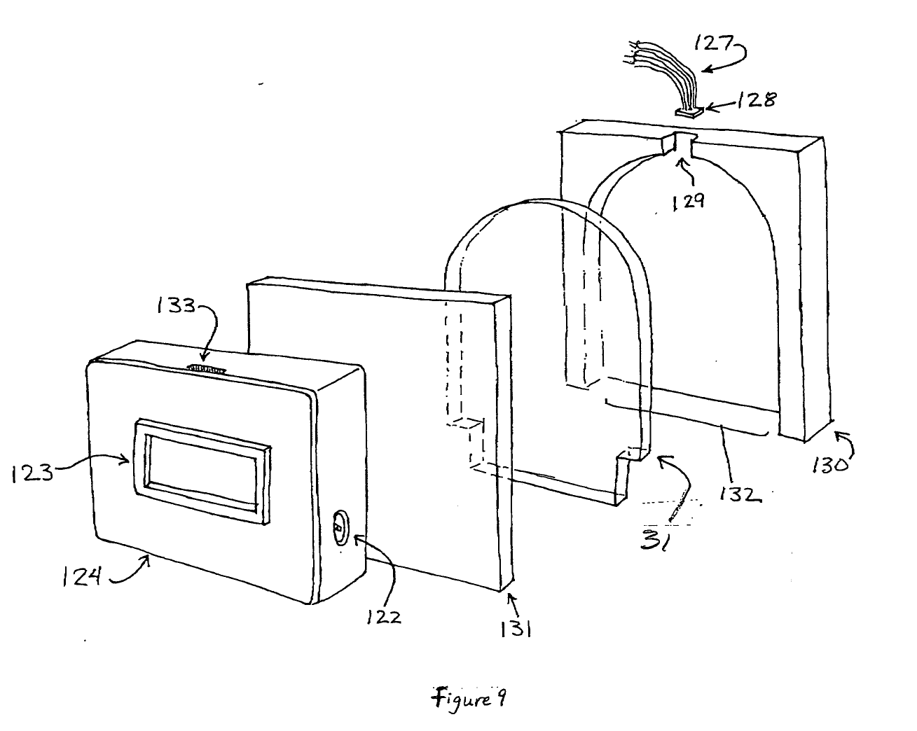

Initially referring to FIGS. 1-10, this invention operates by shining light at/through a subtle-energy producing material. The current exemplary embodiment of this invention uses red, green, and blue colored LEDs 44, 45, and 46 as the light source. These lights flash on and off at a rate that is too fast to be perceived visually (around 130 Hz in the current exemplary embodiment). The duty cycle of the individual lights is varied to produce the impression of the lights becoming brighter and dimmer. By adjusting the duty cycles of the red, green, and blue lights, it is possible to mix other colors of light. As red, green, and blue are the primary colors for light, virtually any color of visible light can be mixed using this scheme. The current exemplary embodiment of this invention has 256 different intensity levels for each color of light. As such, this device may generate over 16.7 million different colors.

It was discovered that polycarbonate has the property of being able to generate a subtle-energy field when light is directed towards and/or through it. The current exemplary embodiment of this invention uses red, green, and blue LEDs 44, 45, and 46, directed at the edge of a sheet of clear polycarbonate being the translucent element 31, to create a subtle-energy field. This embodiment has an LCD display 32 that shows the duty cycle that each color has been set to. Colors of polycarbonate other than transparent should be considered within the scope of this invention. Resolutions other than 8-bit (256 settings per channel) should be considered within the scope of this invention. The use of other kinds and colors of lights, including lights outside of the visible light field (e.g. infrared) should be considered within the scope of this invention. The use of other types of displays should be considered within the scope of this invention, although the use of displays should not be considered mandatory for the use of the present invention.

Lights, such as LEDs, are directed at or through a subtle energy-producing material, such as transparent or translucent polycarbonate in the form of a translucent element 31. The lights may be colored or white, or outside of the visible light spectrum (e.g. infrared). The lights may flash on and off, typically at a frequency that is too fast to be perceived. The lights may also flash from a brighter setting to a dimmer setting, rather than on and off. The waveform of the flashing lights may be square, sinusoidal, or some other form. The duty cycle may be varied. The intensity may be varied. The color of the lights themselves may be varied. The shape of the subtle energy-producing material may vary. Note that a display 32 can be optional, being not absolutely required for use of the present invention.

The present invention may be used in conjunction with biofeedback means, to determine an optimal setting for a particular result. The present invention may also have pre-programmed settings for a variety of purposes. The present invention would also include embodiments that are not adjustable at all.

This exemplary embodiment of the perceptible apparatus 30 comprises a circuit 51 in a housing 33, a translucent element 31 which protrudes from the housing 33, red, green, and blue LEDs 44, 45, 46 in the circuit 51 which direct light at the lower edge of the translucent element 31, an LCD display 32 that provides information about how the perceptible apparatus 30 is operating, adjustment knobs 36, 37, 38, and 39 which control the operation of the perceptible apparatus 30, and a wall-transformer 34 which provides power, and firmware which can be on a microcontroller U1 which controls the operation of this perceptible apparatus 30, thus the use of a microcontroller U1 and its associated firmware is optional, as other methods of providing a signal to the means 64 of producing the variable perceptible output should be considered within the scope of this invention.

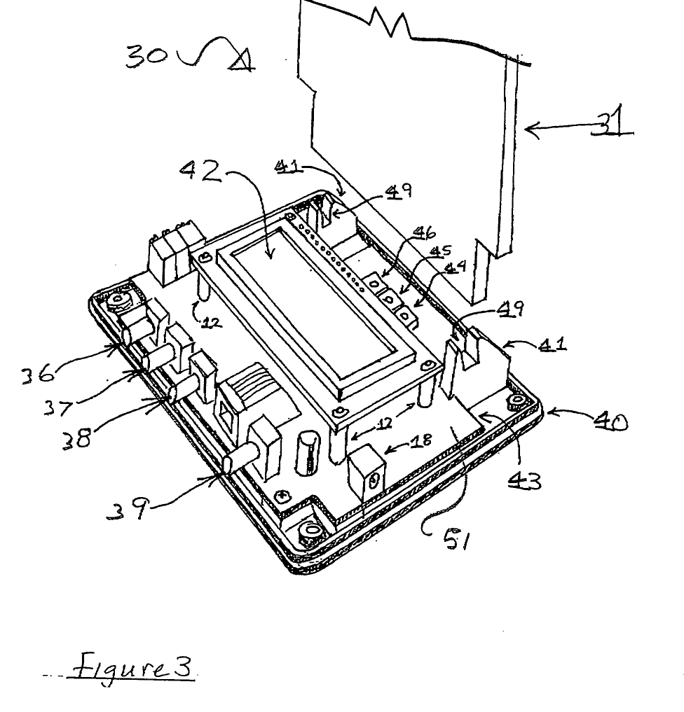

The exemplary embodiment of the perceptible apparatus 30 is for use in helping to create a reactive effect upon a user 54, and includes control circuitry 60 in conjunction with using optional microcontroller U1 and firmware that is operative to generate a one of a plurality of selected signals 62, which could preferably be pulse width modulation signals 62 at a substantially fixed frequency, or could be alternative types of signals 62. A means 64 for producing a variable perceptible output that is in response to one of the plurality of selected pulse width modulation signals 62. The means 64 is preferably the aforementioned LEDs 44, 45, and 46 of the colors red, green, and blue, however, the means 64 could simply be a singular or plurality of lights being preferably LEDs of any color combination or white alone, or anything that is perceptible or create a reactive effect in the user 54. Additionally, a translucent element 31 that is adjacent to the means 64 (as best shown in FIGS. 3, 4, and 6) is for producing a variable perceptible output and/or subtle energy, with the translucent element 31 being operative to diffuse and emit 65 the variable perceptible output/subtle energy. The translucent element 31 is preferably constructed of a synthetic thermoplastic resin and more particularly a polycarbonate, however, alternative materials that transmit a perceptible output or subtle energy would be acceptable. Also, optionally the perceptible apparatus 30 further includes a display 32 that is operational to indicate a relative level of the variable perceptible output.

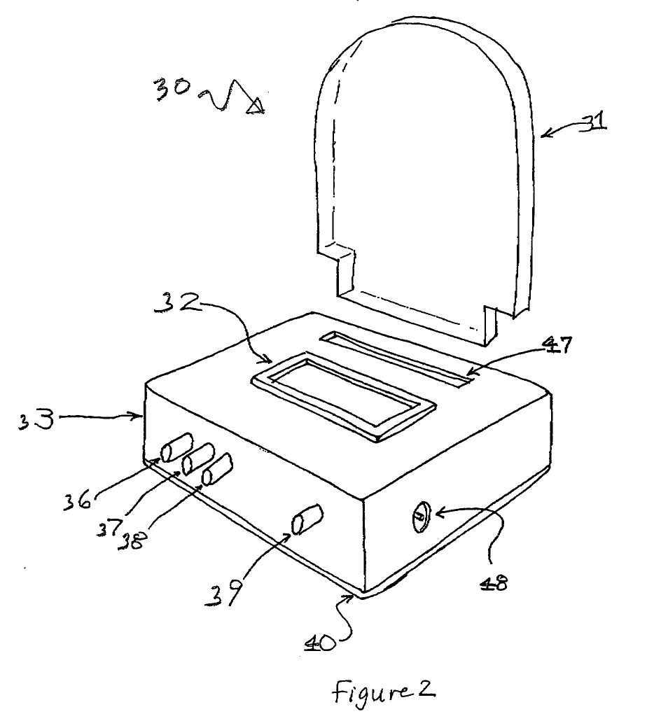

Optionally, an adjacent reflective mirror 66 (as best shown in FIG. 21) can be added that is operational to further control the perceptible output by modifying the diffuse and emit 65 functions of the translucent element 31. Also, it is preferable that the perceptible apparatus control circuitry 60 are contained within a common housing 33 that is substantially parallelepiped in shape (as best shown in FIGS. 1 and 2) or other alternative shapes or configurations as desired for the common housing 33.

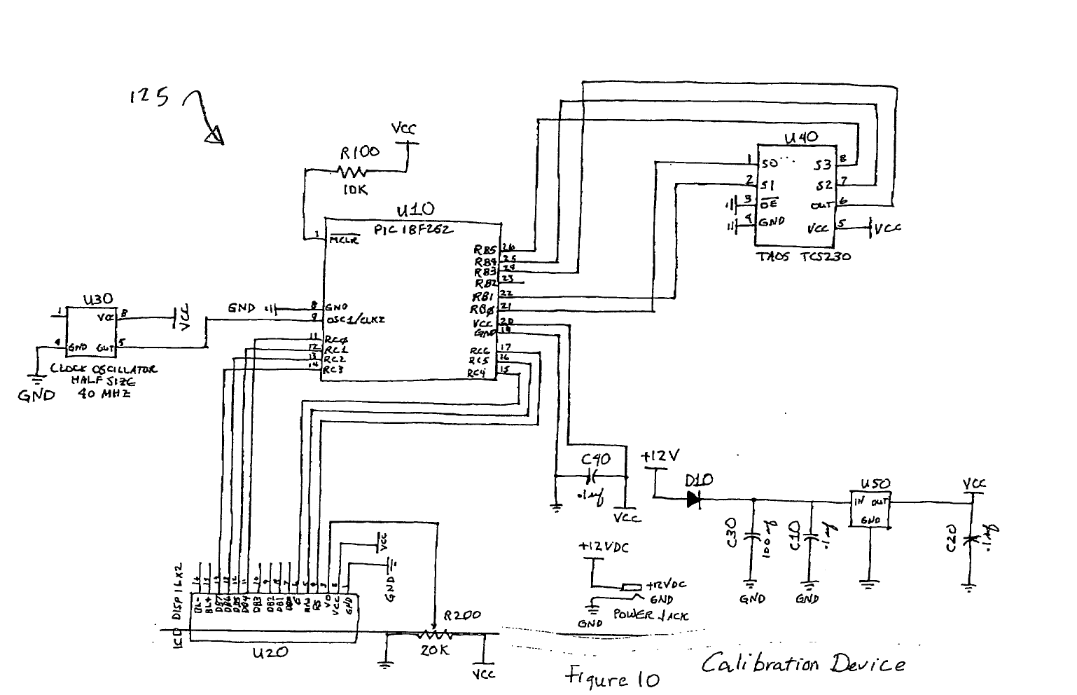

To further enhance or substantially standardize the subtle energy effect the perceptible output of the perceptible apparatus 30 the control circuitry 60 further includes calibration circuitry 68 for the LED of the means 64 for the variable perceptible output that is operational to help make brightness of the LED consistent amongst different LEDs from either different manufacturing batches or different units of the perceptible apparatus 30. To even further enhance or further substantially standardize the subtle energy effect the perceptible output of the perceptible apparatus 30 further includes a calibration device 121 that includes a shroud 126 that occludes substantially all external environment 134 light from the translucent element 31 and calibration device circuitry 125 (as best shown in FIG. 10) that provides a signal that is displayed in a display 123 indicating brightness and/or color of the LED. Brightness of the LED can be adjusted using the calibration device display 123 and the calibration circuitry 68 that is operational to further help make brightness of the LED consistent amongst different LEDs. Color of the LED can be adjusted by using the calibration device display 123 and replacing a selected LED that is operational to further help make color of the LED consistent amongst different LEDs.

As a further option, the perceptible apparatus 30 control circuitry 60 further includes clearing mode functionality selected by circuitry 70 in conjunction with firmware that upon activation is operational to replace the one of a plurality of selected pulse width modulation signals 62 with a continuously changing pulse width modulation signal 62 fluctuating between a maximum and minimum duty cycle with frequency remaining essentially constant.

The exemplary embodiment of the perceptible apparatus 30 for use in helping to create a reactive effect upon a user 54, includes control circuitry 60 operative to generate singularly or simultaneously in each one of a plurality of selected modes a plurality of selected signals 62 preferably being pulse width modulation signals 62 for each said mode. Also included is a plurality of lights being preferably LEDs 44, 45, and 46, with each one light variably illuminated in response to one of a plurality of selected signals 62 preferably being pulse width modulation signals 62 for each one of a plurality of selected modes. Further included is a translucent element 31 adjacent to the plurality of lights being preferably LEDs 44, 45, and 46, the translucent element 31 is operative to diffuse and emit 65 one light illuminated or a plurality of lights illuminated or for subtle energy. Optionally, the perceptible apparatus 30 for the translucent element 31 further includes an adjacent reflective mirror 66 (as best shown in FIG. 21) that is operational to further control the one light illuminated or a plurality of lights illuminated or for subtle energy. The translucent element 31 is preferably constructed of a synthetic thermoplastic resin and more particularly a polycarbonate, however, alternative materials that transmit a perceptible output would be acceptable that meet the aforementioned requirements. Also, it is preferable that the perceptible apparatus 30 control circuitry 60 be contained within a common housing 33 that is substantially parallelepiped in shape (as best shown in FIGS. 1 and 2), or other alternative shapes or configurations as desired for the common housing 33. Also, optionally the perceptible apparatus 30 further includes a display 32 that is operational to indicate a relative illumination level of each of the plurality of LEDs 44, 45, and 46.

To further enhance or for standardizing the energy effect of the perceptible output for the perceptible apparatus 30, the control circuitry 60 further includes calibration circuitry 68 for the LEDs of the means 64 for the variable perceptible output that is operational to help make brightness of the LEDs consistent amongst different LEDs from either the plurality of LEDs in a singular perceptible apparatus 30, different manufacturing batches of LEDs, or different units of the perceptible apparatus 30. To even further enhance or further standardize the energy effect the perceptible output of the perceptible apparatus 30 further includes is a calibration device 121 that includes a shroud 126 that occludes substantially all external environment 134 light from the translucent element 31 and calibration device circuitry 125 (as best shown in FIG. 10) that provides a signal that is displayed in a display 123 indicating brightness and/or color of the LEDs 44, 45, and 46. The brightness of the LEDs 44, 45, and 46 can be adjusted using the calibration circuitry 68 and the display 123 being operational to further help make brightness of the LEDs consistent amongst different LEDs. Color of the LEDs 44, 45, and 46 can be adjusted by using the calibration device display 123 and replacing a selected LED that is operational to further help make color of each one of the LED's consistent amongst the plurality of different LEDs.

It is possible, however, not mandatory to use LEDs that are of the same color, as an example an acceptable range for red is 622-645 NM (nano meters), with the dominant wavelength within 7 NM for blue and green having the dominant wavelength within 7 NM.

As a further option, the perceptible apparatus 30 control circuitry 60 further includes clearing mode functionality selectable by mode select circuitry 70 and/or firmware that upon activation is operational to replace said one of a plurality of selected pulse width modulation signals 62 for each selected mode with a continuous cycle of the red light 44 illuminated to a maximum sequencing to simultaneously reducing illumination of the red light 44 and increasing illumination of the green light 45 to a maximum illumination with the red light 44 not illuminated sequencing to simultaneously reducing illumination of the green light 45 and increasing illumination of the blue light 46 to a maximum illumination with the green light 45 not illuminated sequencing to simultaneously reducing illumination of the blue light 46 and increasing illumination of the red light 44 to a maximum illumination with the blue light 46 not illuminated.

### Description Operation of Exemplary Embodiment of the Calibration Circuit

An important goal of the exemplary embodiment of this invention (as well as many other possible embodiments) is that all perceptible apparatus 30 units will perform consistently with one another. This means that if a perceptible apparatus 30 is set to a particular setting, any other perceptible apparatus 30 could be set to that same setting, and the two perceptible apparatus 30 units would produce substantially identical subtle energy fields. Early in the process of developing this invention, it was believed that the color perceived by the eye was the important thing. As such, the obvious approach to calibration was simply to scale the duty cycles in software, to compensate for variations in the brightness of the LEDs. After some amount of development work, this idea was tested, and found not to work. Even though the colors were identical, the subtle energy fields they produced were very different. As such, it became necessary to devise another calibration scheme.

Through experimentation, it was determined that several factors matter in producing identical subtle energy fields. These factors are: the color of each LED, the brightness of each LED, the duty cycle of the LED, the frequency of pulsation of the LED, and the distance to the translucent element 31. The final three of these factors are easily controlled through engineering. Using a high-quality, bin-sorted LED controls the color of the LEDs. The brightness of the LEDs may be controlled by instituting current-regulating means into the circuit on the perceptible apparatus 30. Being able to calibrate for brightness allows for greater flexibility in selecting bin-sorted LEDs, possibly precluding availability problems when the perceptible apparatus 30 is in production.

After some experimentation, calibration circuitry 68 was developed, which allows adjustment of the current drawn by the LEDs when they are turned on, overcoming differences in brightness between different LEDs. This calibration circuitry 68 performs extremely accurate current sensing, so that use of an alternate power supply will not cause the perceptible apparatus 30 to consume a materially different amount of current. Thus, the perceptible apparatus 30 units will perform consistently, even if the power supplies vary substantially in voltage.

The following narrative describes the operation of the calibration circuit 68. For clarity, only one set of reference designators is (for the blue channel) used in this narrative. Operation is identical in the red, and green channels as well.

Adjustment of the current drawn by the blue LED, being LED 46 is accomplished by controlling the voltage applied to the gate of power MOSFET Q1. The power MOSFET Q1 functions as an adjustable series-resistor, controlling the current through LED1. The calibration circuitry uses an op amp U4 to apply a voltage to the gate of the power MOSFET Q1. One input to the op amp U4 is the PWM signal from the PIC controller U1, scaled through a trimmer potentiometer R19. The other input to the op amp is a signal from a series-resistor R6, in series with LED1 46. This input creates a current-sense signal to the op amp U4. Calibration is accomplished by adjusting the trimmer potentiometer. The circuitry is such that the op amp U4 will try to make the voltages equal at its two inputs. It does this by changing its output, which is applied to the gate of the series MOSFET Q1, until the current-sense voltage is equal to the amplitude-scaled PWM signal from the PIC controller. In this way, the intensity of LED 46 during its on-setting may be controlled by adjusting the trimmer potentiometer R19.

One more nuance deals with the function of resistor R24. Without this resistor, the op-amp maintains enough voltage at the gate of the MOSFET Q1 to keep the LED 46 on slightly, when it is supposed to be off. R24 serves to artificially elevate the voltage of the current-sense input to the op amp U4, especially when the LED is in the “off” state. The result is that the op amp U4 will reduce its output to the gate of the MOSFET Q1 to essentially zero, causing the LED 46 to turn off completely. R24 may cause a slight change in the current flowing through LED 46 in the on state, but this is compensated for in the calibration process.

### Description/Operation of Exemplary Embodiment of the Calibration Device

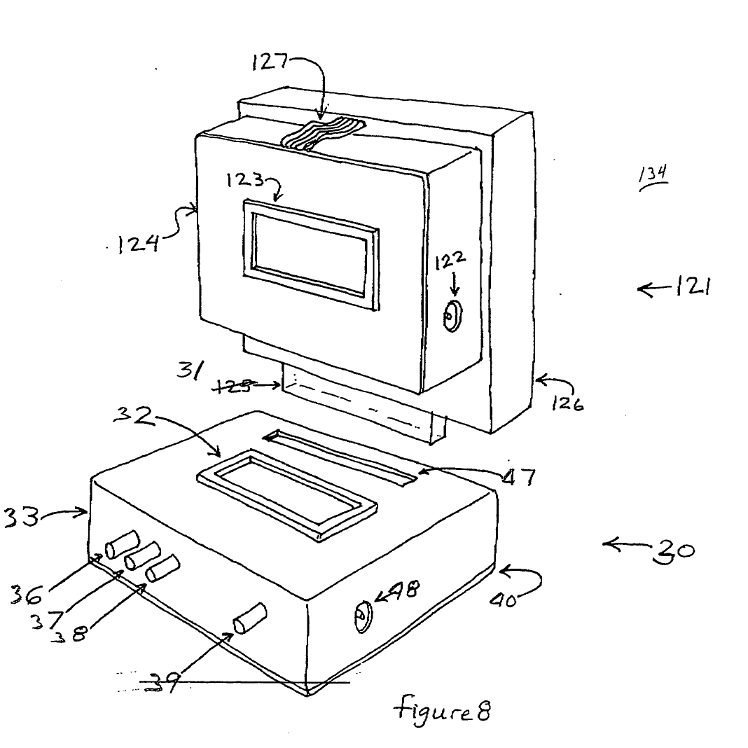

Referring to FIGS. 8-10, once it was known that the perceptible apparatus 30 units could be calibrated by controlling the color and/or adjusting the brightness of the LEDs during the “on” portion of their duty cycle, it was clear that a device was needed for measuring the brightness and/or color of the LEDs. Such a device was built as follows known as a calibration device 121.

A translucent element 31 was fabricated, identical to the translucent element 31 that are part of the perceptible apparatus 30. A black shroud 126 surrounds this translucent element 31, that occludes substantially all external environment light 134 from the translucent element 31. The only portion of the translucent element 31 that is un-shrouded is the part that protrudes into the translucent element slot 47 in the top of the perceptible apparatus 30. In this way, the only significant light that enters the translucent element 31 is the light from the LEDs 44, 45, and 46 themselves.

A slot 129 was cut in the top of the shroud 126, exposing a portion of the top edge of the translucent element 31. A TAOS TCS230 color-sensing chip 128 was placed in this slot 129, exposing it to the light coming from the translucent element 31. In this prototype embodiment, the TCS230 chip 128 was soldered via an adaptor to the ribbon cable 127. The TCS230 chip 128 was then immobilized in the slot with a blob of black RTV. The black RTV serves an additional purpose of sealing out room light that might otherwise enter the slot 129. An alternate design, using more than one TCS230 chip 128 and/or with the TCS230 chip(s) at different locations around the edge of the translucent element 31, should be considered within the scope of this invention. Using other means of mounting the TCS230 chip 128 should also be considered to be within the scope of this invention. Alternatively, other light and/or color measuring means, alternative to the TCS230 chip 128 could be utilized.

The TCS230 chip 128 is interfaced to the microcontroller U10. The TCS230 chip 128 outputs a pulse that increases in frequency, as the light gets brighter. The TCS230 chip 128 has an array of sensors, covered by red, green, blue, and clear filters. The TCS230 chip 128 can be programmed to output pulses corresponding to the brightness of red, green, blue, and the overall brightness of the light detected. In this way, the TCS230 chip gives an accurate measure of the color and/or brightness of the light striking its sensors. The microcontroller U10 simply displays on LCD display 123, the count of these pulses in a particular, arbitrary time period. The LCD display 123 gives four numbers—counts for red, green, and blue, as well as a count corresponding to the clear filter on the TCS230 chip 128. A circuit enclosure 124 containing the microcontroller U10, the LCD display 123, and related circuitry was attached to the shroud 126. The exemplary embodiment of the calibration device 121 is powered by a six-volt to nine-volt wall transformer similar to the one that powers the perceptible apparatus 30. To use the exemplary embodiment of the calibration device 121, the translucent element 31 is removed from the perceptible apparatus 30 being calibrated. The calibration device 121 is inserted in place of the removed translucent element 31. Both devices are powered up. The calibration device 121 immediately begins displaying numbers, which correlate to the brightness of red, green, blue, and white, as detected by the TAOS chip 128. Choose a color, red, green, or blue, and turn its knob 36, 37, or 38 all the way up on the perceptible apparatus 30. The other two color knobs 36, 37, or 38 should be all the way down. The mode-select knob 39 should be in “manual” mode. The LEDs 44, 45, or 46 you have selected should be on steady, at 100 percent duty cycle. The calibration device 121 will display values for red, green, blue, and white. Adjust the trimmer potentiometer R15, R16, or R19, until the proper values are displayed on the calibration device. This adjustment needs to be done fairly quickly, as the LEDs 44, 45, or 46 gradually become less bright as they warm up. When the adjustment is complete, turn that LED's knob 36, 37, or 38 off, and repeat the process with each of the other two LEDs 44, 45, or 46. Once this is complete, the perceptible apparatus 30 should be calibrated. There is one final check that is advisable, however. This is to turn all three color knobs 36, 37, and 38 up all the way. This done, the calibration device 121 will show four numbers which indicate the color (hopefully white) that is produced with all three knobs turned up. This is a final “sanity check,” which is a little bit redundant, and therefore decreases the likelihood of an undetected mistake in the calibration process.

It is also possible that if the LEDs within a batch are consistent enough, calibration could be accomplished by measuring the current drawn by the LEDs, rather than the light emitted. This is assuming that the proper current for this batch of LEDs had been predetermined using the calibration device. In this strategy, the “sanity check” described above (using the calibration device with all colors up full) would likely still be used, to make sure the LEDs are within spec.

### Reference to Computer Source code Program Listing

This application includes a source code computer program termed firmware listing as an attached text file, the contents of which are herein incorporated by reference.

### Method of Use

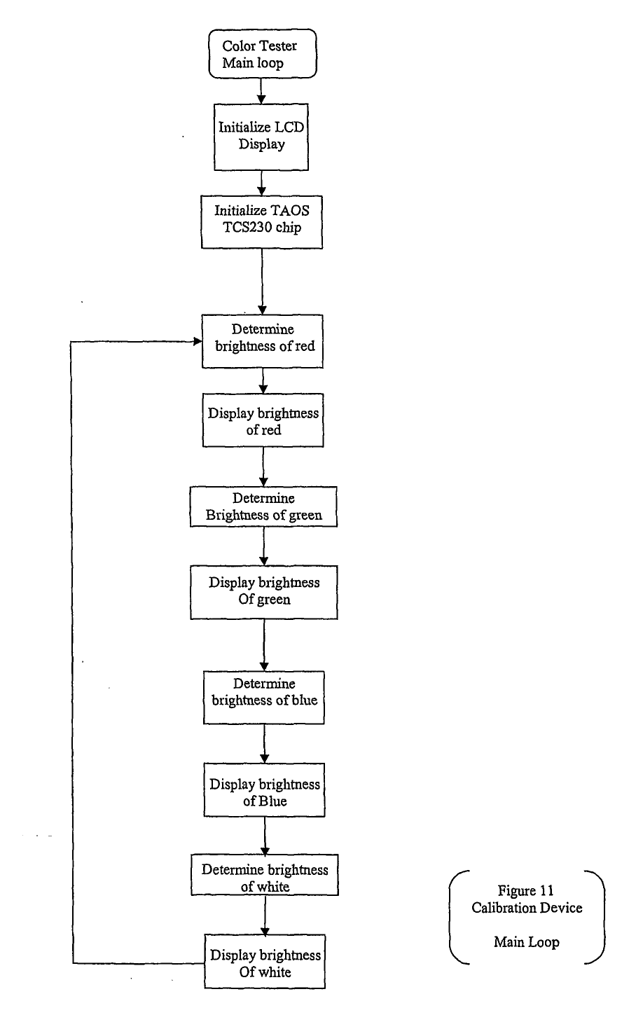

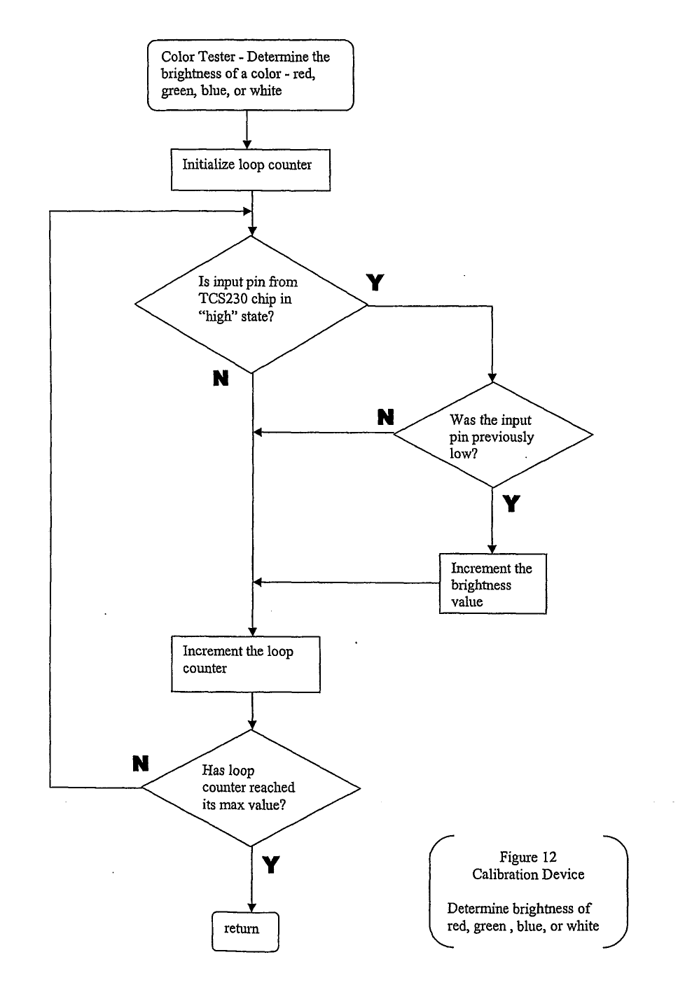

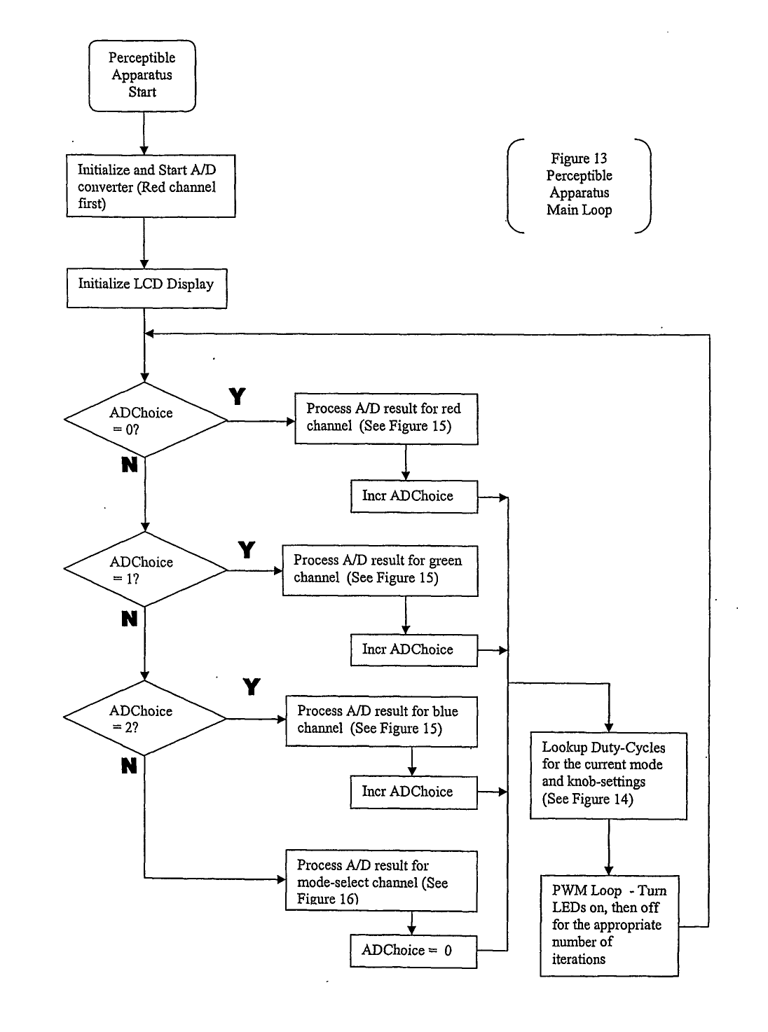

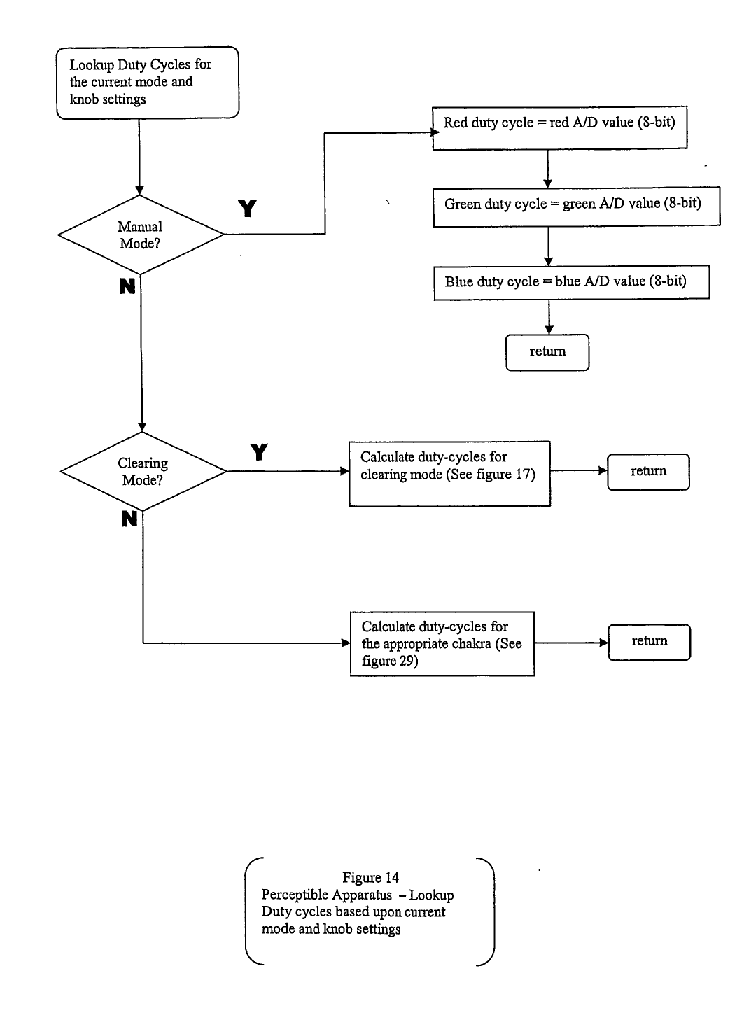

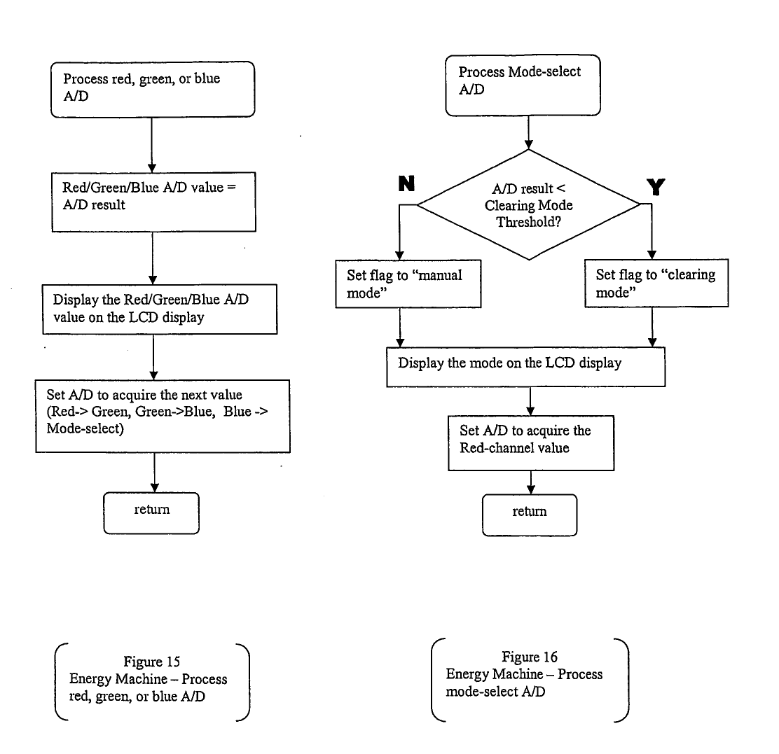

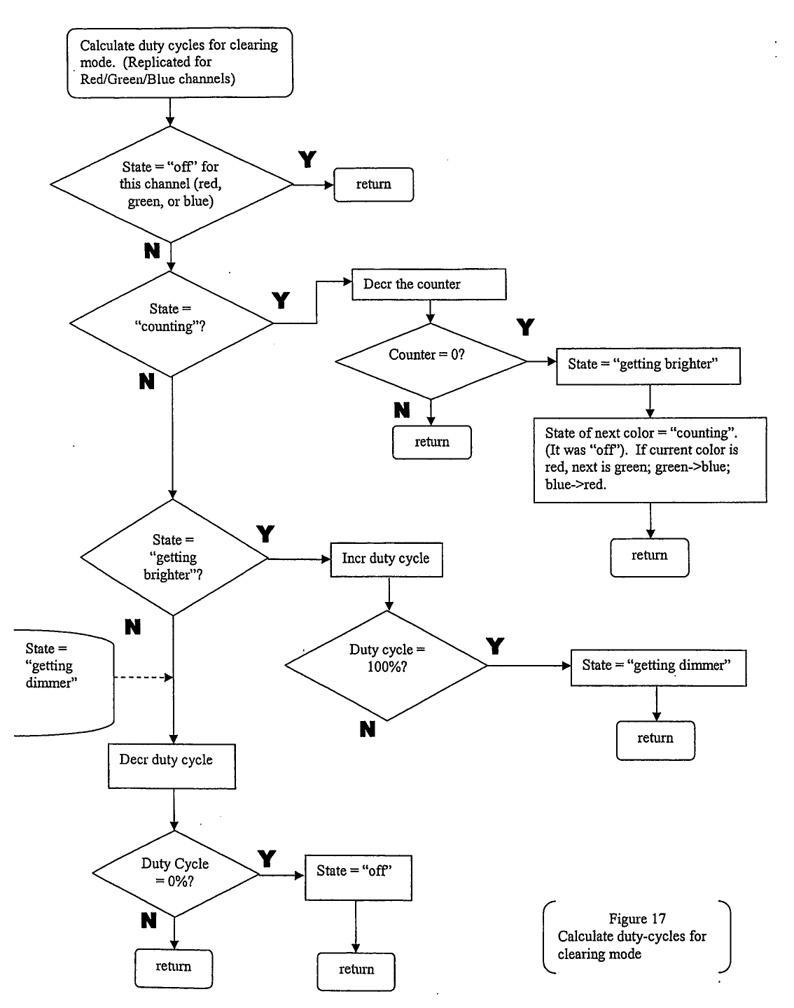

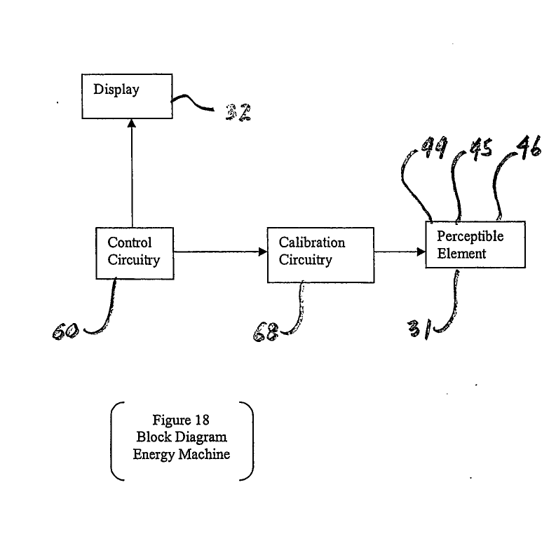

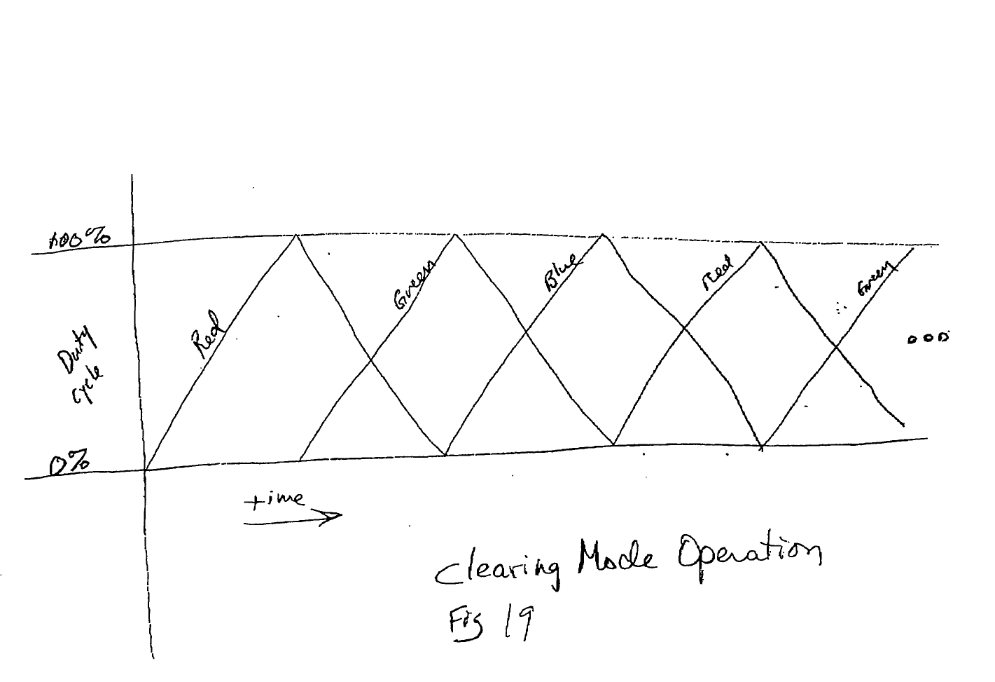

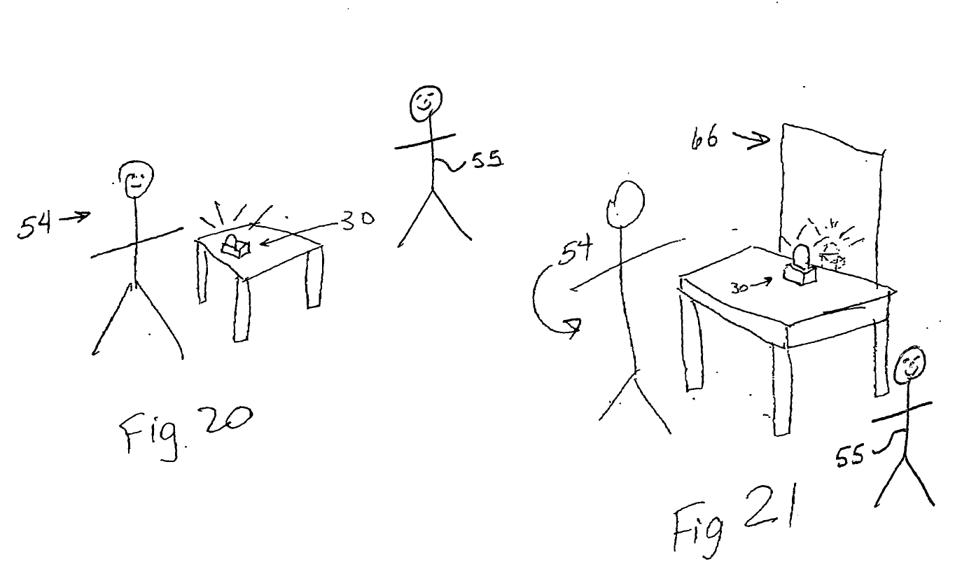

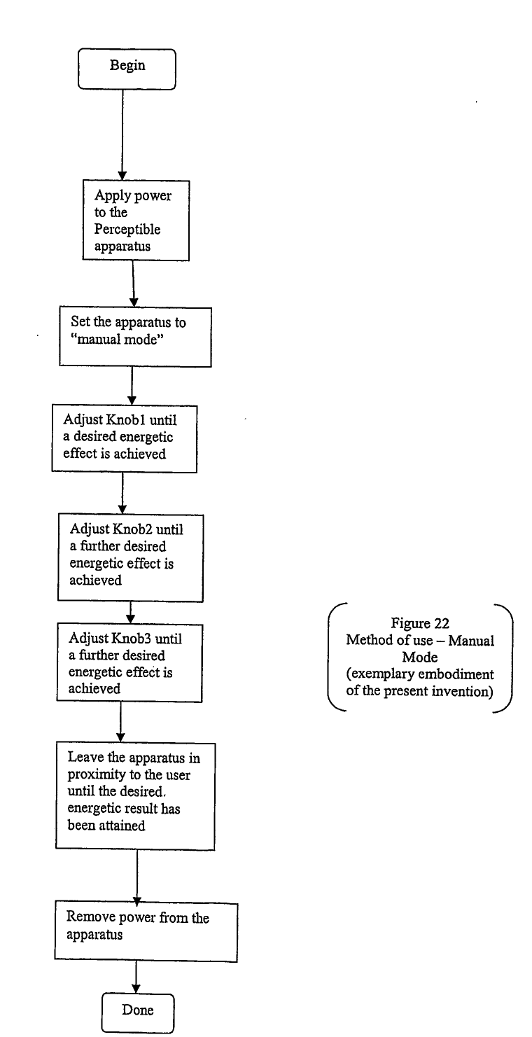

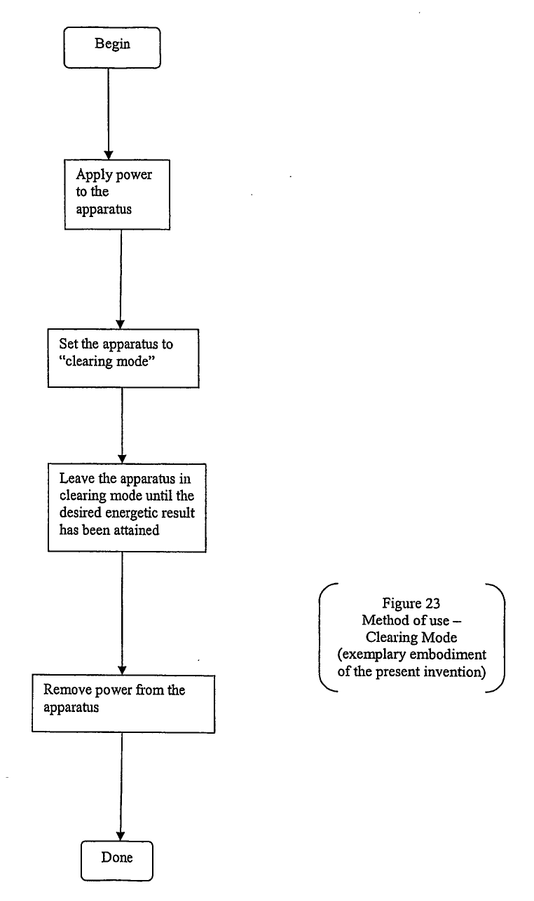

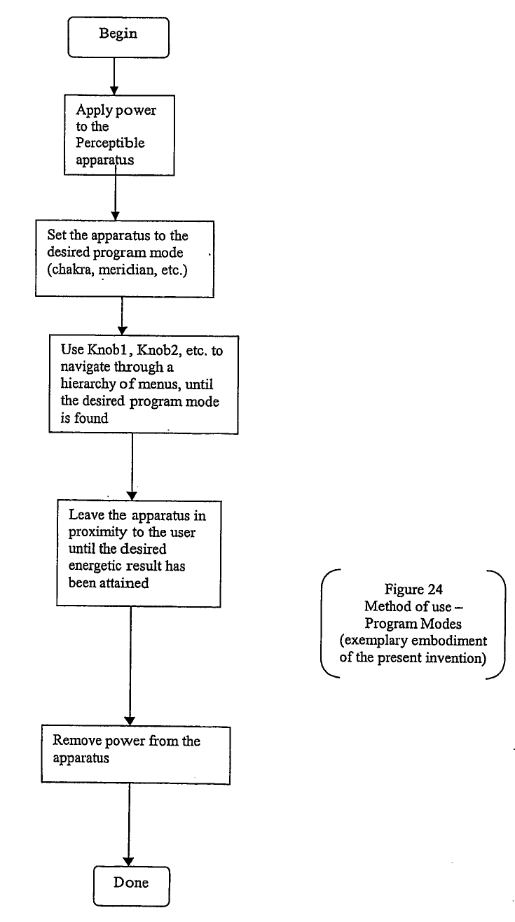

Referring to FIGS. 11-27, the exemplary embodiment of the perceptible apparatus 30 emits a perceptible output and/or subtle energy field, which is tunable, and which enhances and/or resonates with the biofield of the human body. Given the 8-bit resolution over three channels (red, blue, and green), over 16.7 million settings are possible. When the subtle energy field has been tuned to resonate with the energy field of the user 54, a useful/helpful result may be attainable. Tuning is accomplished by adjusting the duty-cycles of the three colors until a resonance occurs between the bioenergetic field of the user 54 and the subtle energy field produced by the perceptible apparatus 30. Rotating the three knobs 36, 37, and 38 makes this adjustment. The existence of this resonance may be determined by the intuition of the practitioner 55, or by the intuition/sensations experienced by the practitioner 55 or user 54. The practitioner 55 being defined as the administrator or controller of the perceptible apparatus 30, wherein the user 54 is seeking the desired reactive or energetic effect with possible help from the practitioner 55. In some cases, the user 54 fulfills the function of the practitioner 55. Alternatively, program modes may be incorporated into the perceptible apparatus 30, which may be used to generate a certain effect, such as balancing a chakra or acupuncture meridian. Additionally, means such as biofeedback may be incorporated, to determine an optimal setting for the perceptible apparatus 30. Either of these two alternatives makes the perceptible apparatus 30 more easily usable by the practitioner 55 or the user 54 who are less gifted intuitively.

### Manual Mode Operation

In the exemplary embodiment of the perceptible apparatus 30, manual mode operation is selected by rotating the “mode-select” knob 39 until the LCD display 32 indicates that you are in manual mode. In manual mode, the three other knobs 36, 37, and 38 take on the roles of adjusting the duty cycles of the red, green, and blue LEDs 44, 45, and 46. The color at the edge of the translucent element 31 changes as these knobs 36, 37, and 38 are adjusted. The display 32 shows a number between 0 and 255 for each color, indicating duty cycle or pulse width modulation signal 62. An intuitive practitioner 55 or user 54 would typically be necessary to set the perceptible apparatus 30 when operating in manual mode. Many practitioners 55 or users 54 are sensitive enough to set the perceptible apparatus 30 for themselves. Some practitioners 55 or users 54 are also sensitive enough to set the perceptible apparatus 30 for use with another practitioner 55 or user 54. A common way to set the perceptible apparatus 30 is to start with all the knobs 36, 37, and 38 turned off. Choose a knob 36, 37, or 38, and slowly turn it until a sense of energetic “resonance” is detected. Do the same with the other two knobs. It is not necessary for the user 54 to look at the perceptible apparatus 30 in order to receive treatment. Only the user 54 merely need to be in the vicinity of the perceptible apparatus 30, a couple of feet away works well for the user's 54 proximity to the perceptible apparatus 30. Sometimes, it works better to have the perceptible apparatus 30 in front of the user 54; at other times, it works better to have the perceptible apparatus 30 behind the user 54. At other times, it does not seem to matter where the perceptible apparatus 30 is.

A user 54 who is not intuitively gifted may use manual mode by having their practitioner determine the settings they should use, for example, at home, between appointments with the practitioner. The user 54 may then reproduce the settings at home by rotating the knobs 36, 37, and 38 until the proper values are displayed on the LCD display 32.

### Program Mode Operation

Program mode operation provides a way of setting the perceptible apparatus 30 appropriately, without requiring the presence of an intuitive practitioner 55 or user 54. Program modes are implemented by incorporating tables into the perceptible apparatus 30 containing predetermined settings. Program modes may be set up to support a variety of modalities. For example, you could have a program mode with table entries mapping to the chakras. A practitioner 55 or user 54 could then easily choose a setting to strengthen a particular chakra energetically. You could also have a program mode focused on acupuncture meridians. Program modes could be set up to energetically support the various organ systems of the body. Additionally, custom program modes could be set up to support many other healing modalities.

Program mode operation is selected by rotating the “mode-select” knob 39 until the desired mode of operation appears on the LCD display 32. In program mode operation, the other knobs 36, 37, and 38, rather than setting colors directly, are used to navigate through a hierarchy of menus to arrive at the desired “canned” selection. The selection may be a single color or it may be a sequence of colors. For example, a program could be created to cycle through the chakras, to balance them. It would also be possible to have a program mode for user-defined settings. This way, a practitioner 55 could program specific settings for a user 54, who could easily reproduce them at home. This might be more convenient than having the user 54 set the settings himself in manual mode.

### Biofeedback Mode Operation

Research needs to be done to determine appropriate biofeedback modalities that reliably indicate that the perceptible apparatus 30 has been properly set for a particular purpose. Early results using the Aurastar 2000 machine appear promising. Assuming such biofeedback modalities are found to be effective, it is strongly desirable to set up a closed-loop system comprising the biofeedback means and the perceptible apparatus 30. In one scenario, the biofeedback means would provide a signal to the perceptible apparatus 30. The perceptible apparatus 30 would try a variety of settings for each color, paying attention to the signal from the biofeedback means. The perceptible apparatus 30 would use this information to determine an optimal setting. In this way, no skill or special intuition would be necessary to use the perceptible apparatus 30 effectively. A simpler variation of this approach would be to have a human operator adjust the perceptible apparatus 30 while observing the output of the biofeedback means. Either approach should be considered to be within the scope of this disclosure.

It appears promising that the early results from testing of the Aurastar 2000 for biofeedback, wherein useful results were provided, thus, it seems likely that other biofeedback methods will be found that will work. This is because the perceptible apparatus 30 works extremely effectively in the hands of an intuitive practitioner. It is clear that the perceptible apparatus 30 is doing something in the body, because a wide variety of physical patterns of discomfort are often improved dramatically in a short period of time. This being the case, it seems very likely that some measurable indicator (probably more than one) will be found which indicates that the perceptible apparatus 30 has been set properly. (Examples are galvanic skin resistance (GSR), heart-rate variability, EEG, EMG, blood pressure, pulse rate, Kirilian photography, and other metrics known to those with skill in the art. Muscle testing has been found to be effective as a means of determining settings for the perceptible apparatus 30.) While the details of this mode of operation are yet to be fleshed out, the idea of using biofeedback as a means to properly set the perceptible apparatus 30 should be considered part of this invention.

### Clearing the Perceptible Apparatus 30

One of the challenges still present is a tendency of the perceptible apparatus 30 to occasionally pick up “stuff.” Often, the perceptible apparatus 30 seems to work by assisting the user 54 in releasing “negativity” from their body's energy field. Sometimes, the perceptible apparatus 30 seems to pick up this negativity, corrupting the subtle energy field produced by the perceptible apparatus 30 in subsequent sessions. The necessity of clearing the perceptible apparatus 30 seems to depend upon the severity of the energetic imbalance being corrected with the perceptible apparatus 30, with a particular user 54.

Sometimes this negativity can be removed from the perceptible apparatus 30 simply by cycling the perceptible apparatus 30 through the full range of red, green, and blue settings. This cycling has been automated by creating a clearing program, selected by the mode-select knob 39. In this clearing mode, the perceptible apparatus 30 fades from red, to green, to blue, and back to red, etc. continuously. The perceptible apparatus 30 cycles through all 1024 possible duty cycle settings for each color. The effect is that the perceptible apparatus 30 cycles through all the colors of the rainbow. One advantage of having an appealing clearing program is the user 54 will likely run the clearing program often, enjoying the pleasant effect it creates. This gives practitioner 55 or user 54 another way of enjoying the perceptible apparatus 30, but more importantly, encourages them to run the clearing mode a lot. It may be that many clearing mode algorithms will be found which work effectively, much as there are many screen-saver programs which work effectively on computers.

Another approach to clearing the perceptible apparatus 30 is to replace the translucent element 31 with a new one. A third approach is to expose the perceptible apparatus 30 to direct sunlight. The balanced, full-spectrum lighting of the sun seems to help clear negative resonance's picked up by the perceptible apparatus 30. A fourth approach to clearing the perceptible apparatus 30 is for a practitioner to use his/her intuition to determine a specific setting of the perceptible apparatus 30 that will clear the negativity. More research needs to be done to determine the best means of clearing the perceptible apparatus 30.

### Use of Clearing Mode as a Program Mode

Interestingly, in addition to clearing perceptible apparatus 30, the clearing program seems to have benefits with respect to balancing the user's 54 energy. As such, the clearing program may be used as a “program mode” as described above. The clearing program does not require any intuitive abilities to operate as a program mode. The clearing program has opened up a whole field of research to be explored, involving symmetrical, changing settings. It is believed that the symmetrical, changing settings are responsible for the clearing effect on the perceptible apparatus 30. Additionally, it is believed that these symmetrical, changing settings will produce useful results without requiring intuitive abilities on the part of the practitioner or user 54. This is because the symmetrical, changing settings cycle through the settings in a balanced way. The effect should be comparable to that of a tape-head demagnetizer, which neutralizes built-up magnetism in a tape head by exposing it to a balanced alternating-current magnetic field.

### Use of the Perceptible Apparatus 30 for Environmental Energetic Clearing

In addition to resonating with the biofield of a human, the perceptible apparatus 30 has potential applications for environmental energetic clearing. Practitioner 55 or user 54 who are sensitive to such things know that subtle energy fields are everywhere. There are a number of devices on the market that claim to emit a subtle energy field that balances harmful effects of subtle energy in the environment. Many of these devices are focused on strengthening a practitioner 55 or user 54 energetically so they are not bothered to the same degree by electromagnetic fields generated by electrical items in the home or workplace. The perceptible apparatus 30 appears to have application as an environmental clearing device. As of this writing, little experimenting has been done in this regard, however the clearing program described above seems to work effectively for environmental clearing.

### Use of Mirrors in Conjunction With the Perceptible Apparatus 30

As of this writing, a small amount of experimentation has been conducted in the use of mirrors 66 in conjunction with the perceptible apparatus 30. It has been observed that mirrors 66 have an effect on the subtle energy field produced by the perceptible apparatus 30. Placing the perceptible apparatus 30 in front of a large, flat mirror 66 tends to spread the energy field, rather than having it more concentrated around the perceptible apparatus 30. Experimentation needs to be done to determine the effects of other shapes of mirrors 66 on the subtle energy field. For example, would a parabolic mirror 66 create a focused beam of subtle energy, much like it does with light? Such an improvement might make it possible to focus a beam on a single user 54, or on a part of their body, without affecting others around them.

### Internal Operation of the Perceptible Apparatus 30

The exemplary embodiment of this invention has three LEDs, colored red 44, green 45, and blue 46. The LEDs 44, 45, and 46 flash at a rate too fast to be seen with the eye. Frequencies between 65 Hz and 260 Hz have been shown to work well. The duty cycle of the flashing LEDs 44, 45, and 46 varies between 0 and 100%, depending upon how the perceptible apparatus 30 has been set. The light produced by these LEDs is directed at the bottom edge of the translucent element 31. Black solder mask was used on the circuit board 43 to prevent the color of the circuit board 43 from influencing the color of the light being directed toward the translucent element 31. Additionally, the inside of the housing 33 is black. The bottom edge of the translucent element 31 is inside of the circuit housing 33. The top and most of the sides of the translucent element 31 are outside of the housing 33 and visible. In operation, the top and sides of the translucent element 31 illuminate with a color determined by the duty cycles of the red, green, and blue LEDs 44, 45, and 46. The perceptible apparatus 30 is controlled by a PIC 18F252 microcontroller U1, made by Microchip.

Four knobs 36, 37, 38, and 39 protrude through the front of the housing 33, and control the operation of the perceptible apparatus 30. These knobs 36, 37, 38, and 39 are part of potentiometers R2, R3, R4, and R5, the output of which is directed to four of the A/D converters in the PIC microcontroller U1. In manual mode, knobs 36, 37, and 38 control the duty cycles of the red, green, and blue LEDs 44, 45, and 46. The PIC microcontroller U1 reads the A/D converters attached to these potentiometers, and uses the resulting value to determine the duty cycle for the corresponding LED 44, 45, or 46. In a program mode, knobs 36, 37, and 38 are used to navigate through a series of menus appropriate to the program mode selected with the mode-select knob 39. This configuration provides a great deal of flexibility, as the user interface may be changed dramatically simply by upgrading the firmware, no hardware changes necessary.

A feature of the exemplary embodiment of this invention is the LCD display 32 on the top of the housing 33. This display 32 gives information about the operation (e.g. current settings) of the perceptible apparatus 30. Another feature is the calibration circuitry 68 incorporated into this embodiment of the invention. This calibration circuitry 68 enables the perceptible apparatus 30 to be calibrated, overcoming differences in brightness between different LEDs. This calibration circuitry 68 performs extremely accurate current sensing, so that use of an alternate power supply will not cause the perceptible apparatus 30 to consume a materially different amount of current.

### Method for Replicating a Desired Energetic Effect

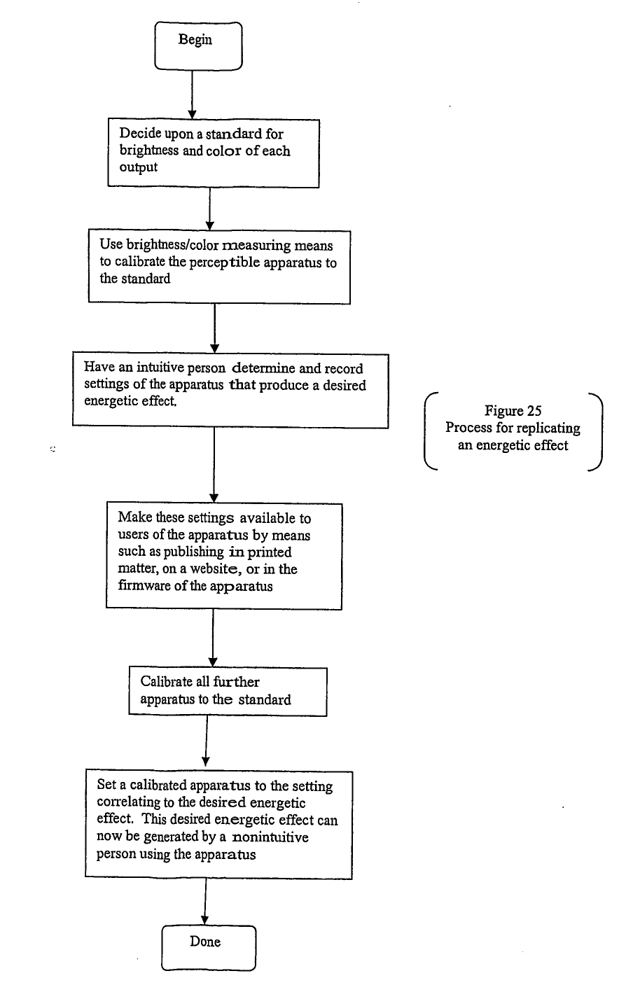

As stated elsewhere in this description, it is strongly desirable for the present invention to produce an energetic effect that is consistent from one perceptible apparatus 30 to the next for any given setting. This foundation of consistency is necessary to implement program modes. Fundamentally, the program modes are a means of replicating an energetic effect that has been deemed desirable. This section describes the overall method of replicating such desirable effects, and is described in FIG. 25.

The first step is to decide upon a calibration standard. The particular standard chosen appears to be somewhat arbitrary. The calibration standard will define the color and brightness of the light sources being LEDs 44, 45, and 46, in their on state. Consistency of color needs to be intrinsic to the light sources themselves in the exemplary embodiment of the perceptible apparatus 30. It has been found that an acceptable range of color is a dominant wavelength varying no more than 23 NM for red, 7 NM for green, and 7 NM for blue.

Once the standard has been defined the perceptible apparatus 30 is calibrated to this standard, using color/brightness measuring means, such as the calibration device 121. This done, the settings necessary to produce desired effects are determined, for example through biofeedback means, or by an intuitive user 54. These setting would typically be used to support a particular modality, such as the chakra system, acupuncture meridians, and the like. These settings would then be made available to all users 54 of the perceptible apparatus 30 within that particular modality. They could then be made available either in program modes in the firmware in the perceptible apparatus 30, or published in some other way, such as printed matter or on a website. Once all this is done, it is possible for practitioner 55 or user 54 who are less intuitively gifted to use the perceptible apparatus 30 effectively to produce an energetic effect.

### Use of the Perceptible Apparatus 30 for Clearing a Crystal

There are a number of holistic modalities that use crystals. It is believed that certain crystals, such as quartz, have subtle energy with desirable qualities. Sometimes however, the subtle energy of a crystal can be corrupted through use in certain holistic modalities. It is possible to use the clearing mode on the perceptible apparatus 30 to clear the energy of the crystal. As shown in FIG. 27, remove the translucent element 31 from the perceptible apparatus 30 and then place the crystal 81 over the slot 47, such that the light from the LEDs 44, 45, and 46 shines through the crystal 81. Then put the perceptible apparatus 30 into the clearing mode, using mode select knob 39. The changing pattern of light generated by the clearing mode will neutralize the energy of the crystal, helping release negative energetic influences the crystal 81 may have acquired.

### Use of the Perceptible Apparatus 30 for Creating Essences

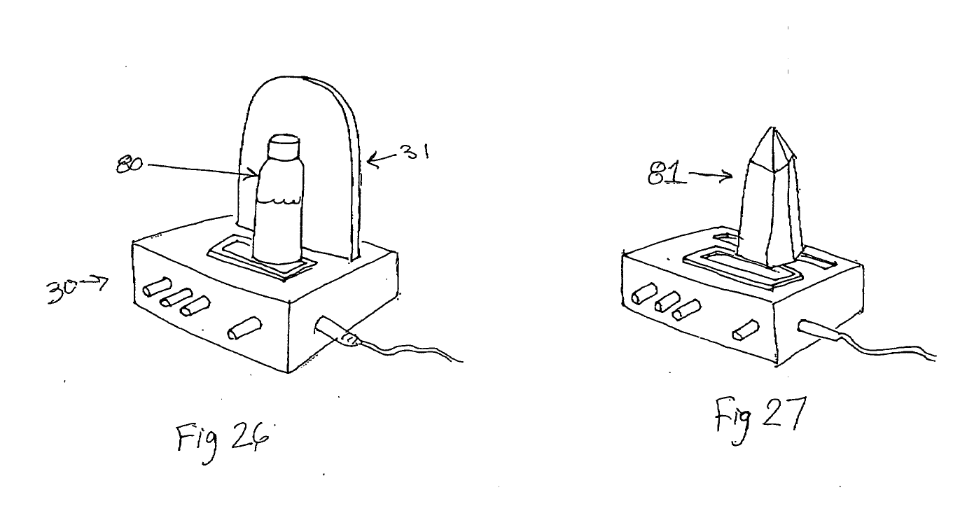

In addition to creating a subtle energy field that works directly on a user 54, perceptible apparatus 30 may be used to encode water or other liquids with a subtle energy. These “essences” that are created with the perceptible apparatus 30 are similar in some ways to homeopathic remedies. To create such an essence, simply set the perceptible apparatus 30 such that it produces a desired energetic effect, then place a small container of water on top of the perceptible apparatus 30 as shown in FIG. 26 adjacent to the translucent element 31. After a period of time, being about 5 minutes or less, the liquid will take on the subtle energy produced by the perceptible apparatus 30.

### Method of Using Balanced, Symmetrical Patterns of Operation for the Perceptible Apparatus 30

Defined as generic “energy balancing” program modes. These modes require no skill or special intuition on the part of the user 54 in using the perceptible apparatus 30. This can be used for energetically clearing the perceptible apparatus 30, a practitioner 55 or user 54 or animal, or an environment.

### Program Mode Example

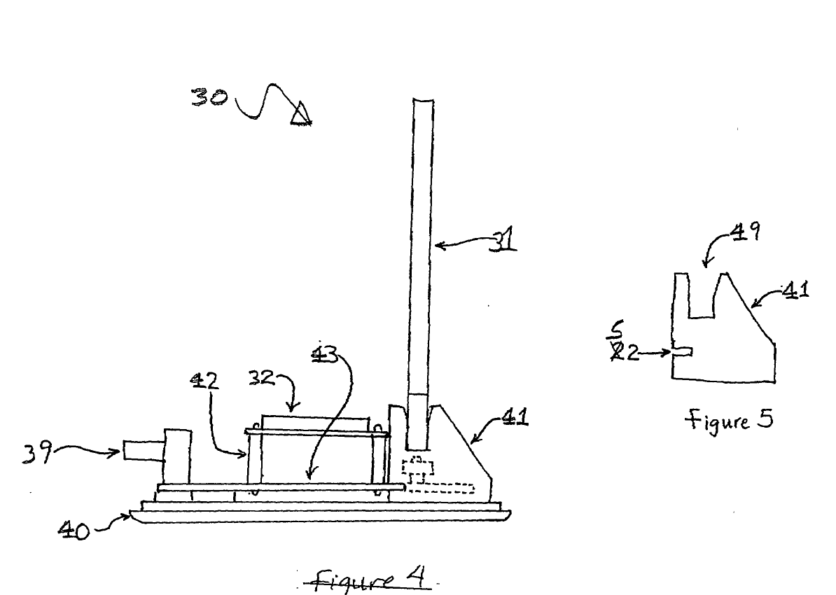

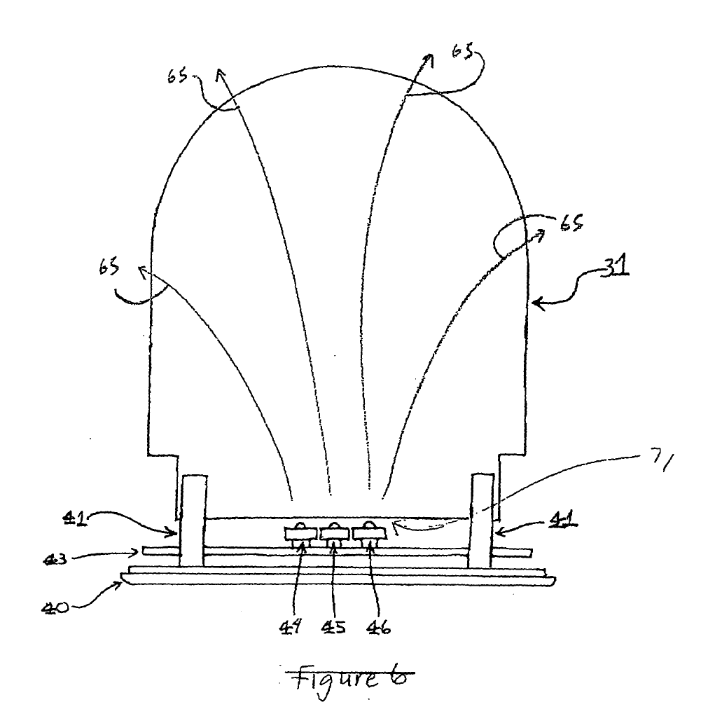

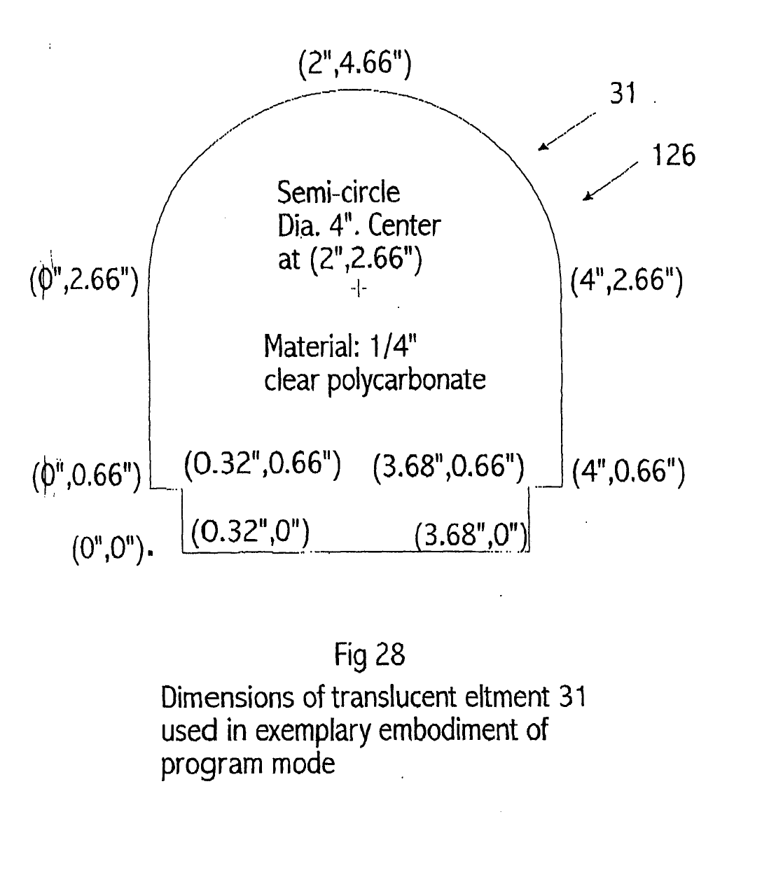

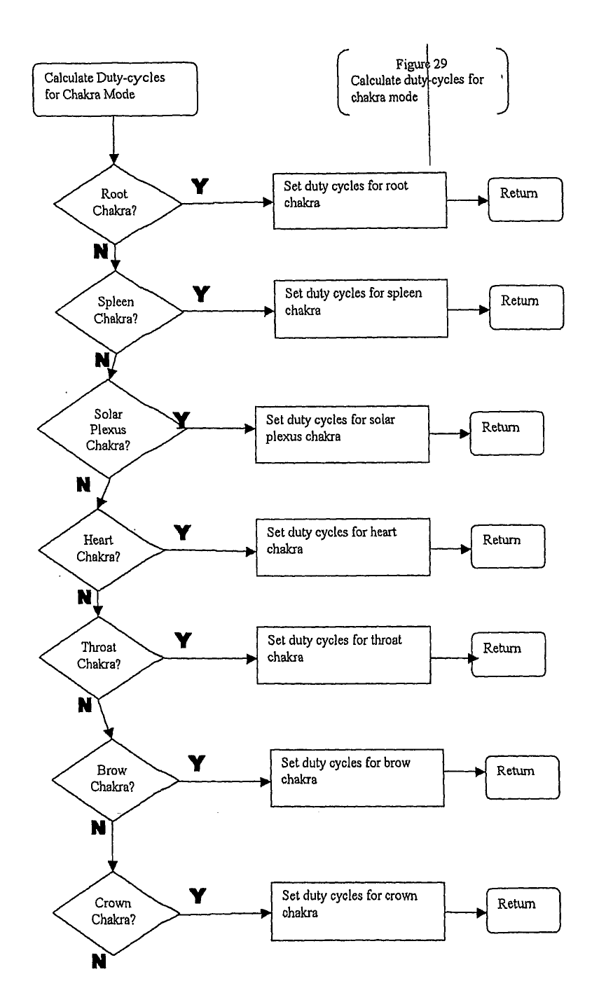

The information is this section is an example of a program mode reduced to practice. In order to make this an enabling disclosure, very specific examples, containing measurements, and the like are provided. This specific information is provided for the purpose of example, not limitation. With the measurements given a setting is provided which will resonate with the root chakra. The necessary measurements for the translucent element 31 in the perceptible apparatus 30 are given in FIG. 28. The measurements are the same for the translucent element 31 in the perceptible apparatus 30 and the translucent element 31 as used in the calibration device 121. In the calibration device 121, the TCS-230 chip 128 is centered at the top of the translucent element 31. The edges of the translucent element 31 are substantially textured, such that they illuminate with a color that represents a relatively uniform mixture of the light emitted by the LEDs 44, 45, and 46. The clearance 72 between the top of the circuit board 43 and the bottom of the translucent element 31 is about 0.28 inches, see FIG. 5 and 6. The LEDs 44, 45, and 46 are centered under the bottom of the translucent element 31 in both the X and Y dimensions, as shown in FIG. 6. The LEDs are all “Superflux” LEDs made by Lumileds. The part No. of the red LED 44 is: HPWT-MD00, category code F04. The part No. for the green LED 45 is: HWPN-MG00, category code G17. The part No. for the blue LED 46 is: HWPN-MB00, category code B26.

The perceptible apparatus 30 is calibrated using the calibration device 121 to give the following brightness/color values: for the red LED 44; Red-9685, Green-217, Blue-101, and White-10,293. For the green LED 45; Red-295, Green-3516, Blue-2578, White-10,293. For the blue LED 46; Red-327, Green-1311, Blue-8454, White-10,761. For consistency's sake, these measurements should be taken immediately after applying power to the LEDs 44, 45, and 46, as the numbers will tend to drop as the LEDs warm up.

In practice the perceptible apparatus 30 is calibrated by focusing on the number corresponding to the color of the LED being calibrated (i.e. to set the brightness of the red LED, adjust potentiometer R15 until the red number displayed 123 on the calibration device 121 is within +/−1% of 9685). The other numbers indicate the particular shade of red, and have an acceptable range of approximately +/−5%. Same process is used for the green LED 45 and the blue LED 46.

Once the perceptible apparatus 30 has been calibrated as described above, the following setting will resonate with the root chakra of a user 54: Red 235, Green 174, and Blue 243. The source code that is attached has a program mode for the root chakra implemented such that if the mode select knob is used to enter “chakra mode” the words “root” and “chakra” will appear on the display 32 and the perceptible apparatus 30 will operate in such a way as to resonate with the root chakra of a user 54.

If the measurements stated above are not followed diligently, the perceptible apparatus 30 will not resonate with the root chakra. However, other standards are possible, and may be equally effective. You would just need a different setting to resonate with the root chakra. With the input of an intuitive practitioner 55 or user 54, or through the use of machine such as the Aurastar 2000 (found to be effective at measuring the effects of the perceptible apparatus 30), other useful settings may be determined, and then replicated on all perceptible apparatus 30 units similarly calibrated.

### Methods of Using and Calibrating the Perceptible Apparatus 30

The method of using the perceptible apparatus 30 for use in helping to create a reactive effect upon a user 54, includes the steps of first positioning the user 54, then providing the perceptible apparatus 30 that includes control circuitry 60 that is operative to generate a one of a plurality of selected signals 62, that are preferably pulse width modulation signals 62, with other signal types acceptable also. Also included in the perceptible apparatus 30 is a means 64 for producing a variable perceptible output in response to one of a plurality of the selected signals 62 and a translucent element 31 adjacent to the means 64 for producing a perceptible output, the translucent element 31 is operative to diffuse and emit the perceptible output. Further a next step is to locate the perceptible apparatus 30 to a selected position adjacent to the user 54. Subsequently, a step of activating the perceptible apparatus 30 being operational to illuminate the means 64 for producing the variable perceptible output that is in response to one of a plurality of the selected signals 62.

A further optional step is of deactivating the perceptible apparatus 30 and reactivating the perceptible apparatus 30 to re illuminate the means 64 for producing the variable perceptible output in response to another one of a plurality of different selected signals 62. Another optional step is of clearing the variable perceptible output 30 that is operational to continuously cycle a brightness of the means 64 for producing the variable perceptible output from a minimum setting to a maximum setting to a minimum setting.

A yet further optional step is to provide a perceptible apparatus 30 that includes control circuitry 60 operative to generate singularly or simultaneously in each one of a plurality of selected modes a plurality of selected signals 62 for each mode, a plurality of different colored lights, preferably being LEDs 44, 45, and 46 with each one light variably illuminated in response to one of a plurality of selected signals 62 for each one of a plurality of selected modes, and a translucent element 31 adjacent to the plurality of lights, the translucent element 31 is operative to diffuse and emit 65 one light illuminated or a plurality of lights illuminated, wherein the activating step initiates a selected sequence to illuminate each of the different colored lights to a selected illumination level.

Another yet further optional step 32 is a step of clearing, wherein the aforementioned different colored lights are operational to continuously cycle an illumination level of each different color light from a minimum setting to a maximum setting sequentially with the different colored lights.

Further a method of calibrating the perceptible apparatus 30 for use in helping to create a reactive effect upon a user, comprises the steps of providing a perceptible apparatus 30 that includes, control circuitry 60 operative to generate singularly or simultaneously in each one of a plurality of selected modes a plurality of selected signals 62 for each mode, a plurality of lights being preferably LEDs 44, 45, and 46, with each one LED variably illuminated in response to one of a plurality of selected signals 62 for each one of a plurality of selected modes, and a translucent element 31 adjacent to the plurality of the LEDs, the translucent element 31 is operative to diffuse and emit 65 one LED illuminated or a plurality of LEDs illuminated, further including calibration circuitry 68 for each one of the LED's that is operational to help make brightness of each one of the LED's consistent amongst the plurality of different LEDs. A further step is to provide a calibration device 121 that includes a shroud 126 that occludes substantially all external environment light 134 from the translucent element 31 and calibration device circuitry 125 that provides a signal that is displayed in a display 123 indicating brightness of the LEDs. Next a step of adjusting brightness using the calibration circuitry 68 and the calibration device display 123 that is operational to further help make brightness of each one of the LED's consistent amongst the plurality of different LEDs.

Yet further a method of calibrating the perceptible apparatus 30 for use in helping to create a reactive effect upon a user, comprises the steps of providing a perceptible apparatus 30 that includes, control circuitry 60 operative to generate singularly or simultaneously in each one of a plurality of selected modes a plurality of selected signals 62 for each mode, a plurality of lights being preferably LEDs 44, 45, and 46, with each one LED variably illuminated in response to one of a plurality of selected signals 62 for each one of a plurality of selected modes, and a translucent element 31 adjacent to the plurality of the LEDs, the translucent element 31 is operative to diffuse and emit 65 one LED illuminated or a plurality of LEDs illuminated, further including calibration circuitry 68 for each one of the LED's that is operational to help make brightness of each one of the LED's consistent amongst the plurality of different LEDs. Subsequently a step of providing a calibration device 121 that includes a shroud 126 that occludes substantially all external environment light 134 from the translucent element 31 and calibration device circuitry 125 that provides a signal that is displayed in a display 123 indicating brightness and color of the LEDs. A next step of adjusting color by using the calibration device 121 and the calibration device display 123 by replacing a selected LED that is operational to further help make color of each one of the LED's consistent amongst the plurality of different LEDs.

### Conclusion

Accordingly, the present invention of a perceptible apparatus has been described with some degree of particularity directed to the embodiment(s) of the present invention. It should be appreciated, though, that the present invention is defined by the following claims construed in light of the prior art so modifications or changes may be made to the exemplary embodiment(s) of the present invention without departing from the inventive concepts contained therein.

1-8. (canceled)

9. A perceptible apparatus for use in helping to create a reactive effect upon a user, comprising:
(a) control circuitry operative to generate a one of a plurality of selected signals;
(b) a light emitting diode (LED) for producing a variable perceptible output in response to said one of a plurality of selected signals, wherein said control circuitry further includes calibration circuitry for said LED that is operational to help make brightness of said LED consistent amongst different LED's: and
(c) a translucent element adjacent to said LED, said translucent element is operative to diffuse and emit said LED variable perceptible output.

10. A perceptible apparatus according to claim 9 further including a calibration device that includes a shroud that occludes substantially all external environment light from said translucent element and calibration device circuitry that provides a signal that is displayed in a display indicating brightness that can be adjusted using said calibration device display and said calibration circuitry that is operational to further help make brightness of said LED consistent amongst different LEDs.

11. A perceptible apparatus according to claim 9 further including a calibration device that includes a shroud that occludes substantially all external environment light from said translucent element and calibration device circuitry that provides a signal that is displayed in a display indicating color that can be adjusted by using said calibration device display and replacing a selected LED that is operational to further help make color of said LED consistent amongst different LEDs.

12-22. (canceled)

23. A perceptible apparatus for use in helping to create a reactive effect upon a user, comprising:
(a) control circuitry operative to generate singularly or simultaneously in each one of a plurality of selected modes a plurality of selected signals for each said mode;
(b) a plurality of light emitting diodes (LEDs), with each one LED variably illuminated in response to said one of a plurality of selected signals for each one of a plurality of selected modes, wherein said control circuitry further includes calibration circuitry for each one of said LED's that is operational to help make brightness of each one of said LED's consistent amongst said plurality of different LED's; and
(c) a translucent element adjacent to said plurality of LED's, said translucent element is operative to diffuse and emit said one LED illuminated or a plurality of LED's illuminated.

24. A perceptible apparatus according to claim 23 further including a calibration device that includes a shroud that occludes substantially all external environment light from said translucent element and calibration device circuitry that provides a signal that is displayed in a display indicating brightness that can be adjusted using said calibration device display and said calibration circuitry that is operational to further help make brightness of each one of said LED's consistent amongst said plurality of different LEDs.

25. A perceptible apparatus according to claim 23 further including a calibration device that includes a shroud that occludes substantially all external environment light from said translucent element and calibration device circuitry that provides a signal that is displayed in a display indicating color that can be adjusted by using said calibration device display and replacing a selected LED that is operational to further help make color of each one of said LED's consistent amongst said plurality of different LEDs.

26-32. (canceled)

33. A method of using a perceptible apparatus for use in helping to create a desired reactive effect upon a user, comprising the steps of:
(a) positioning the user;
(b) providing said perceptible apparatus that includes, control circuitry operative to generate a one of a plurality of selected signals, a means for producing a variable perceptible output in response to said one of a plurality of selected signals, and a translucent element adjacent to said means for producing a variable perceptible output, said translucent element is operative to diffuse and emit said perceptible output;
(c) locating said perceptible apparatus to a selected position adjacent to the user;
(d) activating said perceptible apparatus being operational to illuminate said variable perceptible output in response to one of a plurality of selected signals; and
(e) locating a selected volume of a fluid adjacent to said perceptible apparatus and reinitiating said activating step to create an essence in said fluid with said perceptible apparatus having substantially the same setting as causing the desired reactive effect upon a user.

34. A method of using a perceptible apparatus according to claim 33 further comprising a step of deactivating said perceptible apparatus and reactivating said perceptible apparatus to re illuminate said variable perceptible output in response to another one of a plurality of different selected signals.

35. A method of using a perceptible apparatus according to claim 33 further comprising a step of clearing said variable perceptible output that is operational to continuously cycle a brightness of said variable perceptible output from a minimum setting to a maximum setting to a minimum setting.

36. A method of using a perceptible apparatus according to claim 33 wherein said providing step of a perceptible apparatus includes control circuitry operative to generate singularly or simultaneously in each one of a plurality of selected modes a plurality of selected signals for each said mode, a plurality of different colored lights, with each one light variably illuminated in response to said one of a plurality of selected signals for each one of a plurality of selected modes, and a translucent element adjacent to said plurality of lights, said translucent element is operative to diffuse and emit said one light illuminated or a plurality of lights illuminated, wherein said activating step initiates a selected sequence to illuminate each of said different colored lights to a selected illumination level.

37. A method of using a perceptible apparatus according to claim 36 further comprising a step of clearing, wherein said different colored lights are operational to continuously cycle an illumination level of each different color light from a minimum setting to a maximum setting sequentially with said different colored lights.

38. A method of using a perceptible apparatus for use in helping to create a desired effect upon a crystal, comprising the steps of:
(a) providing said perceptible apparatus that includes control circuitry operative to generate singularly or simultaneously in each one of a plurality of selected modes a plurality of selected signals for each said mode, a plurality of different colored lights, with each one light variably illuminated in response to said one of a plurality of selected signals for each one of a plurality of selected modes, and a translucent element adjacent to said plurality of lights, said translucent element is operative to diffuse and emit said one light illuminated or a plurality of lights illuminated, wherein said activating step initiates a selected sequence to illuminate each of said different colored lights to a selected illumination level;
(b) removing said translucent element from said perceptible apparatus;
(c) locating said plurality of different colored lights to a selected position adjacent to the crystal; and
(d) activating said perceptible apparatus being operational to illuminate said different colored lights that are operational to continuously cycle an illumination level of each different color light from a minimum setting to a maximum setting sequentially with said different colored lights.

39. A method of calibrating a perceptible apparatus for use in helping to create a desired reactive effect upon a user, comprising the steps of:
(a) providing said perceptible apparatus that includes, control circuitry operative to generate singularly or simultaneously in each one of a plurality of selected modes a plurality of selected signals for each said mode, a plurality of LEDs, with each one LED variably illuminated in response to said one of a plurality of selected signals for each one of a plurality of selected modes, and a translucent element adjacent to said plurality of LEDs, said translucent element is operative to diffuse and emit said one LED illuminated or a plurality of LEDs illuminated, further including calibration circuitry for each one of said LED's that is operational to help make brightness of each one of said LED's consistent amongst said plurality of different LEDs;
(b) providing a calibration device that includes a shroud that occludes substantially all external environment light from said translucent element and calibration device circuitry that provides a signal that is displayed in a display indicating brightness of said LEDs; and
(c) adjusting brightness using said calibration circuitry and said calibration device display that is operational to further help make brightness of each one of said LED's consistent amongst said plurality of different LEDs.

40. A method of calibrating a perceptible apparatus for use in helping to create a desired reactive effect upon a user, comprising the steps of:
(a) providing said perceptible apparatus that includes, control circuitry operative to generate singularly or simultaneously in each one of a plurality of selected modes a plurality of selected signals for each said mode, a plurality of LEDs, with each one LED variably illuminated in response to said one of a plurality of selected signals for each one of a plurality of selected modes, and a translucent element adjacent to said plurality of LEDs, said translucent element is operative to diffuse and emit said one LED illuminated or a plurality of LEDs illuminated, further including calibration circuitry for each one of said LED's that is operational to help make brightness of each one of said LED's consistent amongst said plurality of different LEDs;
(b) providing a calibration device that includes a shroud that occludes substantially all external environment light from said translucent element and calibration device circuitry that provides a signal that is displayed in a display indicating color of said LEDs; and
(c) adjusting color by using said calibration device and said calibration device display by replacing a selected LED that is operational to further help make color of each one of said LED's consistent amongst said plurality of different LEDs.

41. A method of replicating a desired reactive effect to further consistency of the desired reactive effect from one perceptible apparatus to another perceptible apparatus, comprising the steps of:
(a) providing a plurality of said perceptible apparatuses that each include, control circuitry operative to generate singularly or simultaneously in each one of a plurality of selected modes a plurality of selected signals for each said mode, a plurality of LEDs, with each one LED variably illuminated in response to said one of a plurality of selected signals for each one of a plurality of selected modes, and a translucent element adjacent to said plurality of LEDs, said translucent element is operative to diffuse and emit said one LED illuminated or a plurality of LEDs illuminated, further including calibration circuitry for each one of said LED's that is operational to help make brightness of each one of said LED's consistent amongst said plurality of different LEDs;
(b) providing a calibration device that includes a shroud that occludes substantially all external environment light from said translucent element and calibration device circuitry that provides a signal that is displayed in a display indicating brightness and color of said LEDs;
(c) selecting a calibration standard for a color from one said perceptible apparatus to another said perceptible apparatus;
(d) selecting a calibration standard for a brightness from one said perceptible apparatus to another said perceptible apparatus;
(e) calibrating color and brightness of a single perceptible apparatus to said color calibration standard and to said brightness calibration standard;
(f) determining a setting on said plurality of perceptible apparatuses that have been calibrated to said color and brightness standards which cause said desirable reactive effect upon the user; and
(g) providing said setting to other users of said brightness and color calibrated perceptible apparatuses.

42. A method of using a perceptible apparatus for use in helping to create a desired reactive effect upon a user, comprising the steps of:
(a) positioning the user;
(b) providing said perceptible apparatus that includes, control circuitry operative to generate a one of a plurality of selected signals, a means for producing a variable perceptible output in response to said one of a plurality of selected signals, and a translucent element adjacent to said means for producing a variable perceptible output, said translucent element is operative to diffuse and emit said perceptible output;
(c) locating the perceptible apparatus to a selected position adjacent to the user;
(d) activating said perceptible apparatus being operational to illuminate said variable perceptible output in response to one of a plurality of selected signals;
(e) determining a setting on said perceptible apparatus which causes said desirable reactive effect upon the user; and
(f) programming said setting in said perceptible apparatus to allow the user to substantially replicate said desirable reactive affect.

43. A method of using a perceptible apparatus for use in helping to create a desired reactive effect upon a user utilizing a biofeedback system, comprising the steps of:
(a) positioning the user;
(b) providing said perceptible apparatus that includes, control circuitry operative to generate a one of a plurality of selected signals, a means for producing a variable perceptible output in response to said one of a plurality of selected signals, and a translucent element adjacent to said means for producing a variable perceptible output, said translucent element is operative to diffuse and emit said perceptible output;
(c) providing said biofeedback system;
(d) locating the perceptible apparatus to a selected position adjacent to the user;
(e) connecting said biofeedback system to said perceptible apparatus and to said user;
(d) activating said perceptible apparatus being operational to illuminate said variable perceptible output in response to one of a plurality of selected signals; and
(e) determining a setting on said perceptible apparatus which causes said desirable reactive effect upon the user based upon a biofeedback system output.
